(19) United States
(12) Patent Application Publication
(10) Pub. No.: US 2003/0134846 A1
(43) Pub. Date: Jul. 17, 2003

(54) Treatment of trypanosoma brucei with farnesyl protein transferase inhibitors

(75) Inventors: William T. Windsor, East Brunswick, NJ (US); Patricia C. Weber, Yardley, PA (US); Corey Strickland, Martinsville, NJ (US); Rosalinda Syto, Nutley, NJ (US); Viyyoor M. Girijavallabhan, Parsippany, NJ (US); James J. Kaminski, Long Valley, NJ (US); Zhuyan Guo, Scotch Plains, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/266,036

(22) Filed: Oct. 7, 2002

Related U.S. Application Data
(60) Provisional application No. 60/327,934, filed on Oct. 9, 2001.

(51) Int. Cl. A61K 31/5513; A61K 31/496; A61K 31/4709

(52) U.S. Cl. 514/221; 514/312; 514/253.03

(57) ABSTRACT

Disclosed is a method of treating and or preventing infections of Trypanosoma brucei by administering to a patient, in need of such treatment, an effective amount of a Farnesyl Protein Transferase Inhibitor alone or in combination with an additional anti-Trypanosoma brucei agent and/or an anti-Trypanosoma brucei resistance reversing agent.

This application claims the benefit of priority of U.S. Serial No. 60/327,934 filed Oct. 9, 2001. The present invention relates to a method of treating Trypanosoma brucei infections, comprising administering an effective amount of a Farnesyl Protein Transferase inhibitor to a patient in need of such treatment alone or in combination with an additional anti-Trypanosoma brucei agent.

### FIELD OF THE INVENTION

The present invention also relates to methods of preparing such inhibitors, compositions containing such inhibitors and methods of using such inhibitors in the treatment of Trypanosoma brucei infections.

### BACKGROUND

The disease commonly known as sleeping sickness is caused by the parasite Trypanosoma brucei. This disease, which is transmitted to humans through the bite of the tsetse fly, has been estimated to affect 300-500 thousand people and maybe fatal if left untreated.

Trypanosoma brucei exists as two subspecies, Trypanosoma brucei rhodesiense and Trypanosoma brucei gambiense. Each form develops within the tsetse fly and the metacyclic trypomastigote of each form is passed into the bloodstream of its victim through the salivary gland of the fly following a bite. Once in the blood stream the parasite spreads rapidly throughout the host. The initial infection stage involves infection of the lymph system, and results in enlarged lymph nodes, headache and irregular fevers. The second stage involves invasion of the central nervous system with neurological symptoms of progressive mental apathy, extended daytime sleeping, and loss of appetite. These neurological effects along with concurrent involvement of the muscular system can result in progression to paralysis and irreversible coma.

The parasite's ability to invade the central nervous system has required two separate treatments due to the difficulty of drug penetration through the blood-brain barrier. Each of the current treatments requires drug delivery through injections. Intramuscular injections are required for either pentamidine isethionate or suramine sodium for the early stage of infection. The second stage of infection, involving the central nervous system, requires intravenous injections of either melarsoprol or eflornithine. These drugs result in serious side effects including, hypotension, abdominal pain, vertigo, hypersalivation and mild nephrotoxicity for pentamindine isethionate treatment and nausea, vomiting, uticaria and possible renal damage or exfoliative dermatitis when using suramine sodium. The development of new drugs for treating sleeping sickness has been very slow. For example, the commonly used second stage drug melarsoprol was developed in 1932 and is a highly toxic arsenic-based molecule. This drug can cause myocardial damage, hypertension, exfoliative dermatitis and reactive encephalopathy, which occurs in 5-10% of the patients and can lead to death. Eflornithine, an inhibitor of the enzyme ornithine decarboxylase, is the only drug suitable for patients where melarsoprol is ineffective but it is poorly effective against Trypanosome brucei rhodesiense. This drug causes mild side effects such as diarrhea, anemia, thrombocytopenia, vomiting and fever.

Recently, Trypanosoma brucei resistant strains to these drugs have been identified. For this reason and due to the few treatment options it is important to develop new therapeutic strategies for treating this disease. In an effort to identify a new and more effective method for treating Trypanosoma brucei Gelb et al. (Yokoyama, et al., Molecular and Biochemical Parasitology, 87, 61-69,1997; Yokoyama et al., Molecular and Biochemical Parasitology, 94, 87-97, 1998; Yokoyama, et al., J. Biological Chemistry, 272, 26497-26505,1998) reported the successful use of prenyl transferase inhibitors in inhibiting the growth of Trypanosoma brucei parasites.

In view of the need to find new treatments for Trypanosoma brucei infections, those skilled in the art would welcome an effective method for the treatment utilizing inhibitors of Trypanosoma brucei prenyl transferases.

### SUMMARY OF THE INVENTION

This invention provides a method of treating and or preventing infections of Trypanosoma brucei by administering to a patient, in need of such treatment, an effective amount (e.g., a therapeutically effective amount, or an effective amount to inhibit Trypanosoma brucei) of a Farnesyl Protein Transferase Inhibitor selected from the compounds A-U shown below.

| COMPOUND # | STRUCTURE |
|---|---|
| A | 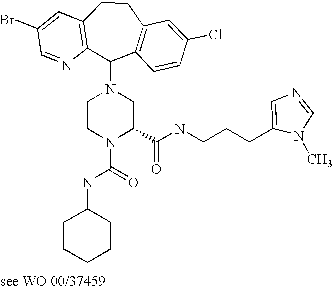 |
| B | 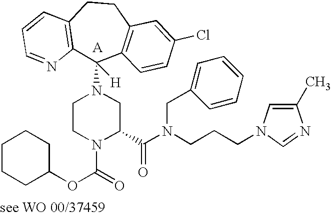 |
| C | 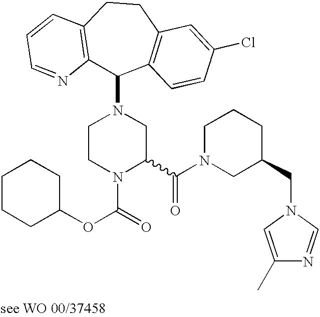 |
| D | 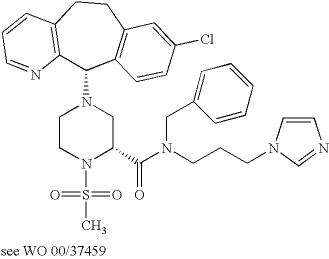 |
| E | 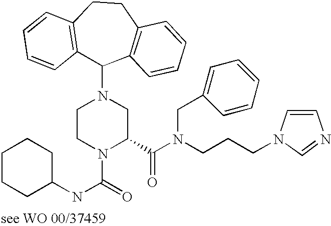 |
| F | 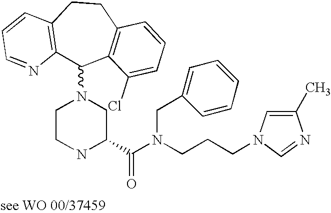 |
| G | 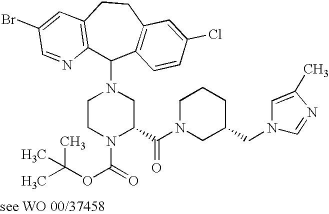 |
| H | 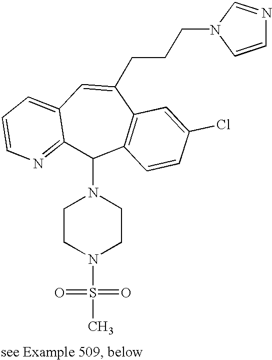 |
| I | 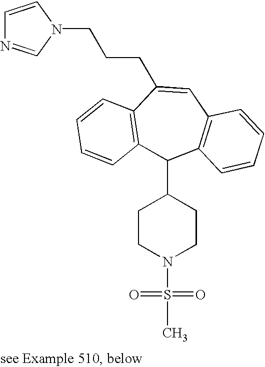 |
| J | 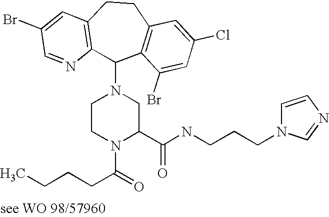 |
| K | 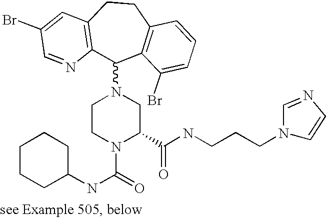 |
| L | 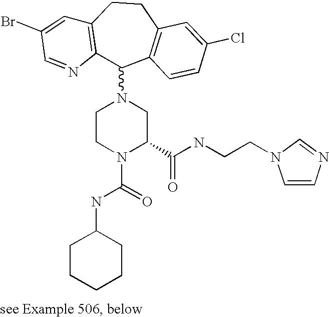 |
| M | 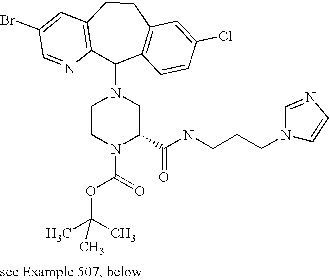 |
| N | 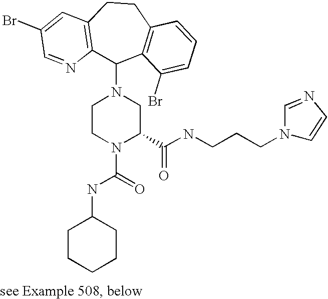 |
| O | 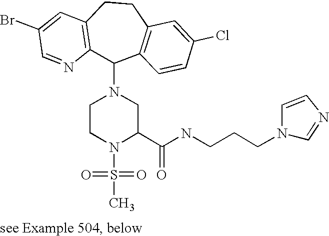 |
| P | 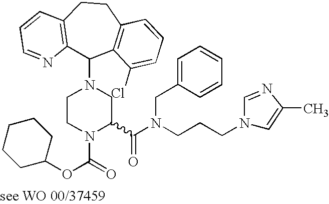 |
| Q | 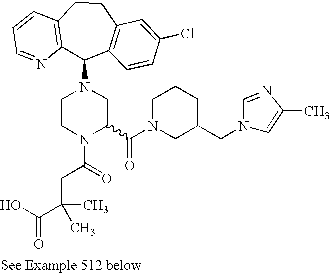 |
| R | 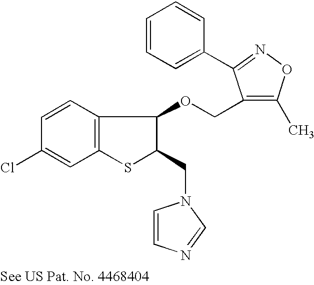 |
| S | 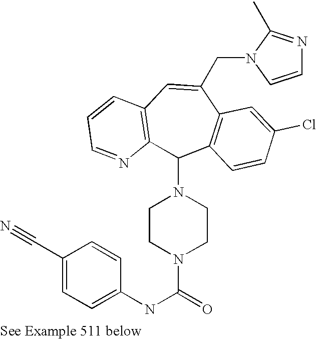 |
| T | 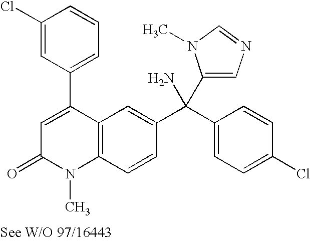 |
| U | 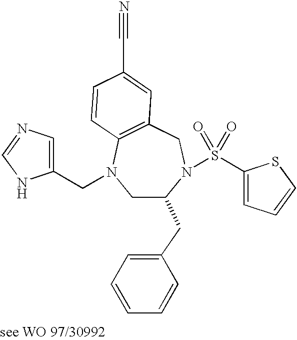 |

In another embodiment, this invention provides a method of treating and/or preventing Trypanosoma brucei infections comprising administering to a patient, in need of such treatment, an effective amount (e.g., a therapeutically effective amount, or an amount to inhibit Trypanosoma brucei infection) of a compound of the formulas I, II, III, IV and V described below.

In yet another embodiment, this invention provides a method of treating Trypanosoma brucei infections, wherein, the compounds A-U shown herein below, are administered in combination with an additional anti-Trypanosoma brucei agent and/or an anti-Trypanosoma brucei resistance reversing agent.

In still another embodiment, this invention provides a method of treating Trypanosoma brucei infections, wherein, a compound of the formulas I, II, III, IV and V described herein below, is administered in combination with an additional anti-Trypanosoma brucei agent and/or an anti-Trypanosoma brucei resistance reversing agent.

### DETAILED DESCRIPTION

One skilled in the art will appreciate that the positions of the Nitrogen atoms around the imidazole portion of the compounds described below are:

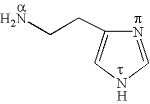

In one embodiment, this invention provides a method of treating and/or preventing Trypanosoma brucei infections comprising administering to a patient, in need of such treatment, an effective amount (i.e., a therapeutically effective amount, or an amount to inhibit Trypanosoma brucei infection) of a compound of the formulas I, II, III, and IV described below.

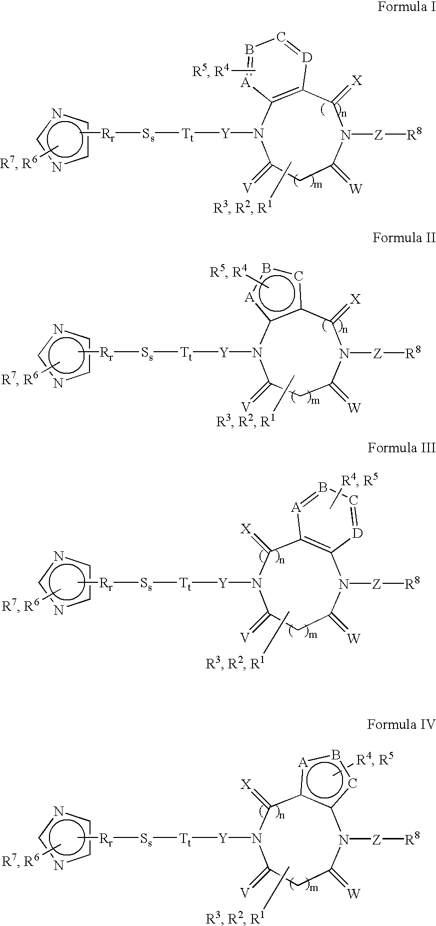

or their pharmaceutically acceptable salts or solvates thereof, wherein:

m, n, r, s and t are 0 or 1;

p is 0, 1 or 2;

V, W and X are selected from the group consisting of oxygen, hydrogen, R¹, R² or R³;

Z and Y are selected from the group consisting of CHR⁹, SO₂, SO₃, CO, CO₂, O, NR¹⁰, SO₂NR¹¹, CONR¹²,

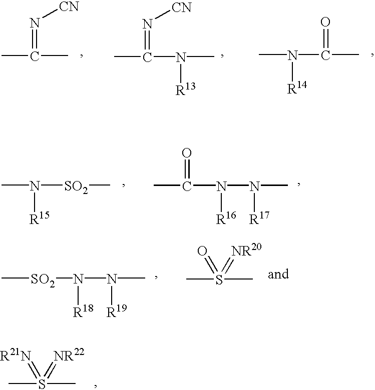

or Z may be absent;

R⁶, R⁷, R⁹, R¹⁰, R¹¹, R¹², R¹³, R¹⁴, R¹⁵, R¹⁶, R¹⁷, R¹⁸, R¹⁹, R²⁰, R²¹, R²², R²⁴, R²⁵, R²⁶, R²⁷, R²⁸, R²⁹, R³⁰, R³¹, R³², R³³, R³⁴, R³⁵, R³⁶, R³⁷, and R³⁸ are selected from the group consisting of hydrogen, lower alkyl, substituted alkyl, aryl, or substituted aryl;

R⁴, R⁵ are selected from the group consisting of hydrogen, halo, nitro, cyano and U-R²³;

U is selected from the group consisting of sulfur, oxygen, NR²⁴, CO, SO, SO₂, CO₂, NR²⁵CO₂, NR²⁶CONR²⁷, NR²⁸SO₂, NR²⁹SO₂NR³⁰, SO₂NR³¹, NR³²CO, CONR³³, PO₂R³⁴ and PO₃R³⁵ or U is absent;

R¹, R², and R³ are selected from the group consisting of hydrogen, alkyl, alkoxycarbonyl, substituted alkyl, alkenyl, substituted alkenyl, alkynyl, substituted alkynyl, aralkyl, cycloalkyl, aryl, substituted aryl, heterocyclo, substituted heterocyclo, cyano, carboxy, carbamyl (e.g. CONH₂) or substituted carbamyl further selected from CONH alkyl, CONH aryl, CONH aralkyl or cases where there are two substituents on the nitrogen selected from alkyl, aryl or aralkyl; R⁸ and R²³ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo, substituted heterocyclo;

any two of R¹, R² and R³ can be joined to form a cycloalkyl group;

R, S and T are selected from the group consisting of CH₂, CO and CH(CH₂)pQ wherein Q is NR³⁶R³⁷, OR³⁸, or CN; and

A, B, C and D are carbon, oxygen, sulfur or nitrogen with the provisos that

1. when m is zero then V and W are not both oxygen or

2. W and X together can be oxygen only if Z is either absent, O, NR¹⁰, CHR⁹,

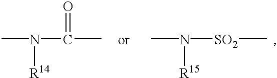

in formulas I and II, and V and X together can be oxygen only if Y is O, NR¹⁰, CHR⁹,

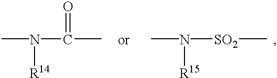

in formulas III and IV or

2. R²³ may be hydrogen except when U is SO, SO₂, NR²⁵CO₂ or NR²⁸SO₂, or

4. R⁸ may be hydrogen except when Z is SO₂, CO₂, or

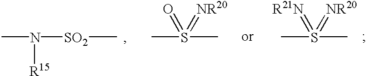

R³⁹ is halo, trifluoromethyl, trifluoromethoxy, hydroxy, alkoxy, cycloalkoxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl; alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido, substituted sulfonamido, nitro, cyano, carboxy, CON(R⁴⁴)₂, where the two R⁴⁴ moieties can be the same or different, alkoxycarbonyl, aryl, substituted aryl, guanidine or heterocyclo;

R⁴⁰ is halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino or heterocyclo;

R⁴¹ is halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino or heterocyclo;

R⁴² is alkyl, substituted alkyl, halo, trifluoromethoxy, trifluoromethyl, hydroxy, alkoxy, cycloalkoxy, heterocyclooxy, alkanoyl, alkanoyloxy, amino, alkylamino, aralkylamino, cycloalkylamino, heterocycloamino, dialkylamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido or aryloxy;

R⁴³ is alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different each being independently selected from the group consisting of hydrogen, alkyl, aryl, and aralkyl; and

R⁴⁴ is alkyl, aryl or aralkyl.

In another embodiment, this invention provides a method of treating and/or preventing Trypanosoma brucei infections comprising administering to a patient, in need of such treatment, an effective amount (i.e., a therapeutically effective amount, or an amount to inhibit Trypanosoma brucei infection) of a compound of the formula V described below.

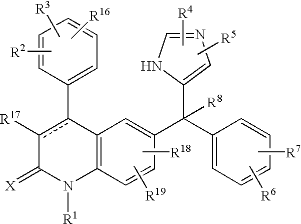

or a pharmaceutically acceptable salt or solvate thereof

wherein:

the dotted line represents an optional bond;

X is oxygen or sulfur;

R¹ is hydrogen, alkyl, Ar¹, Ar²alkyl, quinolinylalkyl, pyridylalkyl, hydroxyalkyl, alkoxyalkyl, aminoalkyl; or a radical of formula -Alk¹-C(═O)—R⁹, -Alk¹-S(O)—R⁹ or -Alk¹-S(O)₂—R⁹, wherein Alk¹ is alkanediyl, R⁹ is hydroxy, alkyl, alkoxy, amino, alkylamino or alkylamino substituted with alkoxycarbonyl;

R², R³ and R¹⁶ each independently are hydrogen, hydroxy, halo, cyano, alkyl, alkoxy, hydroxyalkoxy, alkoxyalkoxy, aminoalkoxy, Ar¹, Ar²alkyl, Ar²oxy, Ar²alkoxy, hydroxycarbonyl, alkoxycarbonyl, trihalomethyl, trihalomethoxy, alkenyl, 4,4-dimethyloxazolyl; or when on adjacent positions R² and R³ taken together may form a bivalent radical of formula

—O—CH₂—O—

—O—CH₂—CH2-O—

—O—CH═CH—

—O—CH₂—CH₂

—O—CH₂—CH₂—CH₂

or

—CH═CH—CH═CH—

R⁴ and R⁵ each independently are hydrogen, halo, Ar¹, alkyl, hydroxyalkyl, alkoxyalkyl, alkoxy, alkylthio, amino, hydroxycarbonyl, alkoxycarbonyl, alkylS(O)alkyl or alkylS(O)₂ alkyl with the proviso that when R⁴ or R⁵ is bound to one of the nitrogen atoms in the imidazole ring, the hydrogen on the nitrogen is replaced by R⁴ or R⁵ wherein R⁴ and R⁵ is selected from the group consisting of hydrogen, Ar¹, alkyl, hydroxyalkyl, alkoxyalkyl, alkoxycarbonyl, alkylS(O)alkyl and alkylS(O)₂alkyl;

R⁶ and R⁷ each independently are hydrogen, halo, cyano, alkyl, alkoxy, Ar²oxy, trihalomethyl, alkylthio, alkylamino, or when on adjacent positions R⁶ and R⁷ taken together may form a bivalent radical of formula —O—CH₂—O— or —CH═CH—CH═CH—;

R⁸ is hydrogen, alkyl, cyano, hydroxycarbonyl, alkoxycarbonyl, alkylcarbonylalkyl, cyanoalkyl, alkoxycarbonylalkyl, carboxyalkyl, hydroxyalkyl, aminoalkyl, imidazolyl, haloalkyl, alkoxyalkyl, aminocarbonylalkyl, or a radical of formula —O—R¹⁰, —S—R¹⁰, —N—R¹¹R² wherein

R¹⁰ is hydrogen, alkyl, alkylcarbonyl, Ar¹, Ar²alkyl, alkoxycarbonylalkyl, or a radical or formula -Alk²-OR¹³ or -Alk²-NR¹⁴R¹⁵;

R¹¹ is hydrogen, alkyl, Ar¹ or Ar²alkyl;

R¹² is hydrogen, alkyl, alkylcarbonyl, alkoxycarbonyl, alkylaminocarbonyl, Ar¹, Ar²alkyl, alkylcarbonylalkyl, an amino acid, Ar¹carbonyl, Ar²alkylcarbonyl, aminocarbonylcarbonyl, alkoxyalkylcarbonyl, hydroxy, alkoxy, aminocarbonyl, di(alkyl)aminoalkylcarbonyl, amino, alkylamino, alkylcarbonylamino, or a radical or formula -Alk²-OR¹³ or -Alk²-NR¹⁴R¹⁵;

wherein

Alk² is alkanediyl;

R¹³ is hydrogen, alkyl, alkylcarbonyl, hydroxyalkyl, Ar¹ or Ar²alkyl;

R¹⁴ is hydrogen, alkyl, Ar¹ or Ar²alkyl;

R¹⁵ is hydrogen, alkyl, alkylcarbonyl, Ar¹ or Ar²alkyl;

R¹⁷ is hydrogen, halo, cyano, alkyl, alkoxycarbonyl, Ar¹;

R¹⁸ is hydrogen, alkyl, alkoxy or halo;

R¹⁹ is hydrogen or alkyl;

Ar¹ is phenyl or phenyl substituted with alkyl, hydroxy, amino, alkoxy or halo; and

Ar² is phenyl or phenyl substituted with alkyl, hydroxy, amino, alkoxy or halo.

As used above, and throughout the specification, the following terms, unless otherwise indicated, shall be understood to have the following meanings:

“Patient” includes both human and other mammals.

“Mammal” means humans and other animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl, n-butyl, and t-butyl.

“Alkenyl” means an aliphatic hydrocarbon group containing at least one carbon-carbon double bond and-which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The term “substituted alkenyl” means that the alkenyl group may be substituted by one or more substituents which may be the same or different, each substituent being independently selected from the group consisting of halo, alkyl, aryl, cycloalkyl, cyano, and alkoxy. Non-limiting examples of suitable alkenyl groups include ethenyl, propenyl, n-butenyl, and 3-methylbut-2-enyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, and 2-butynyl. The term “substituted alkynyl” means that the alkynyl group may be substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be unsubstituted or optionally substituted on the ring with one or more substituents which may be the same or different, each being independently selected from the group consisting of alkyl, aryl, heteroaryl, aralkyl, alkylaryl, 1-5 aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different each being independently selected from the group consisting of hydrogen, alkyl, aryl, and aralkyl. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted on the ring by replacing an available hydrogen on the ring by one or more substituents which may be the same or different, each being independently selected from the group consisting of alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different, each being independently selected from the group consisting of hydrogen, alkyl, aryl, and aralkyl. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrrolyl, triazolyl, and the like.

The term “substituted alkyl” refers to an alkyl group substituted by, for example, one to four substituents, such as, halo, trifluoromethyl, trifluoromethoxy, hydroxy, alkoxy, cycloalkoxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl; alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido, e.g. SO₂NH₂, substituted sulfonamido, nitro, cyano, carboxy, carbamyl, e.g. CONH₂, substituted carbamyl e.g. CONH alkyl, CONH aryl; CONH aralkyl or cases where there are two substituents on the nitrogen selected from alkyl, aryl or aralkyl; alkoxycarbonyl, aryl, substituted aryl, guanidino and heterocyclos, such as, indolyl, imidazolyl, furyl, thienyl, thiazolyl, pyrrolidyl, pyridyl, pyrimidyl and the like. Where noted above where the substituent is further substituted it will be with halogen, alkyl alkoxy, aryl or aralkyl.

The term “substituted aryl” refers to an aryl group substituted by, for example, one to four substituents such as alkyl, substituted alkyl, halo, trifluoromethoxy, trifluoromethyl, hydroxy, alkoxy, cycloalkoxy, heterocyclooxy, alkanoyl, alkanoyloxy, amino, alkylamino, aralkylamino, cycloalkylamino, heterocycloamino, dialkylamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido, aryloxy and the like. The substituent may be further substituted by halo, hydroxy, alkyl, alkoxy, aryl, substituted aryl, substituted alkyl or aralkyl.

The term “substituted alkenyl” refers to an alkenyl group substituted by, for example, one to two substituents, such as, halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino and heterocyclo, e.g. indolyl, imidazolyl, furyl, thienyl, thiazolyl, pyrrolidyl, pyridyl, pyrimidyl, and the like.

The term “substituted alkynyl” refers to an alkynyl group substituted by, for example, a substituent, such as, halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino and heterocyclo, e.g. imidazolyl, furyl, thienyl, thiazolyl, pyrrolidyl, pyridyl, pyrimidyl and the like.

The term “heterocyclo” refers to an optionally substituted, fully saturated or unsaturated, aromatic or nonaromatic cyclic group, for example, which is 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2, 3, or 4 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom.

“Aralkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthlenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting examples of suitable alkylaryl groups include o-tolyl, p-tolyl and xylyl. The bond to the parent moiety is through the aryl.

“Alkylheteroaryl” means a group derived from a fused heteroarylalkyl as defined herein by removal of a hydrogen atom from the heteroaryl portion. Non-limiting examples of suitable alkylheteroaryls are as described herein for heteroarylcycloalkyl, except that the bond to the parent moiety is through an aromatic carbon atom.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted on the ring by replacing an available hydrogen on the ring by one or more substituents which may be the same or different, each being independently selected from the group consisting of alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-. Y₁Y₂NC(O)— and Y₁Y₂NSO₂—, wherein Y₁ and Y₂ may be the same or different each being independently selected from the group consisting of hydrogen, alkyl, aryl, and aralkyl. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkoxy” means a cycloalkyl-O— group in which the cycloalkyl group is as previously described. Non-limiting examples of suitable alkoxy groups include cyclobutoxy, cyclopropoxy, cyclopentoxy and cyclohexoxy. The bond to the parent moiety is through the ether oxygen.

“Cycloalkylamino” means a cycloalkyl-amino group in which the cycloalkyl group is as previously described. The bond to the parent moiety is through the amino.

“Halo” means fluoro, chloro, bromo, or iodo groups. Preferred are fluoro, chloro or bromo, and more preferred are fluoro and chloro.

“Halogen” means fluorine, chlorine, bromine, or iodine. Preferred are fluorine, chlorine or bromine, and more preferred are fluorine and chlorine.

“Haloalkyl” means an alkyl as defined above wherein one or more hydrogen atoms on the alkyl is replaced by a halo group defined above.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted on the ring by replacing an available hydrogen on the ring by one or more substituents which may be the same or different, each being independently selected from the group consisting of alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different each being independently selected from the group consisting of hydrogen, alkyl, aryl, and aralkyl. Non-limiting examples of suitable monocyclic cycloalkenyls include cyclopentenyl, cyclohexenyl, cycloheptenyl, and the like. Non-limiting example of a suitable multicyclic cycloalkenyl is norbornylenyl.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted on the ring by replacing an available hydrogen on the ring by one or more substituents which may be the same or different, each being independently selected from the group consisting of alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different each being independently selected from the group consisting of hydrogen, alkyl, aryl, and aralkyl. The nitrogen or sulfur atom of the heterocyclenyl can be optionally oxidized to the corresponding N-oxide, S-oxide or S,S-dioxide. Non-limiting examples of suitable monocyclic azaheterocyclenyl groups include 1,2-dihydropyridyl, 1,4-dihydropyridyl, 1,2,3,6-tetrahydropyridyl and the like. Non-limiting examples of suitable oxaheterocyclenyl groups include 3,4-dihydro-2H-pyran, dihydrofuranyl, and the like. Non-limiting example of a suitable multicyclic oxaheterocyclenyl group is 7-oxabicyclo[2.2.1]heptenyl. Non-limiting examples of suitable monocyclic thiaheterocyclenyl rings include dihydrothiophenyl, dihydrothiopyranyl, and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are-no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. The heterocyclyl can be optionally substituted on the ring by replacing an available hydrogen on the ring by one or more substituents which may be the same or different, each being independently selected from the group consisting of alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different each being independently selected from the group consisting of hydrogen, alkyl, aryl, and aralkyl. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl and the like.

“Heterocycloamino” means a heterocyclo-amino- group in which the heterocyclo group is as previously described. The bonding to the parent moiety is through the amino group

“Heterocyclooxy” means a heterocyclo-O— group in which the heterocyclo group is as previously described. The bonding to the parent moiety is through the O group.

“Aralkenyl” means an aryl-alkenyl- group in which the aryl and alkenyl are as previously described. Preferred aralkenyls contain a lower alkenyl group. Non-limiting examples of suitable aralkenyl groups include 2-phenethenyl and 2-naphthylethenyl. The bond to the parent moiety is through the alkenyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, 2-(furan-3-yl)ethyl and quinolin-3-ylmethyl. The bond to the parent moiety is through the alkyl.

“Heteroaralkenyl” means an heteroaryl-alkenyl- group in which the heteroaryl and alkenyl are as previously described. Preferred heteroaralkenyls contain a lower alkenyl group. Non-limiting examples of suitable heteroaralkenyl groups include 2-(pyrid-3-yl)ethenyl and 2-(quinolin-3-yl)ethenyl. The bond to the parent moiety is through the alkenyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)—, alkenyl-C(O)—, Alkynyl-C(O)—, cycloalkyl-C(O)—, cycloalkenyl-C(O)—, or cycloalkynyl-C(O)— group in which the various groups are as previously described. The bond to the parent moiety is through the carbonyl. Preferred acyls contain a lower alkyl. Non-limiting examples of suitable acyl groups include formyl, acetyl, propanoyl, 2-methylpropanoyl, and cyclohexanoyl.

“Alkanoyl” means an alkane-C(O)— group in which the alkyl group is as previously described. The bond to the parent moiety is through the carbonyl.

“Aralkanoyl” means aralkyl-C(O)— group in which the aralkyl group is as previously described. The bond to the parent moiety is though the carbonyl.

“Alkanoylamino” means an alkanoyl-amino- group in which the alkanoyl group is as previously described. The bond to the parent moiety is through the amino.

“Alkanoyloxy” means an alkanoyl-O— group in which the alkanoyl group is as previously described. The bond to the parent moiety is through the oxygen.

“Aroyl” means an aryl-C(O)— group in which the aryl group is as previously described. The bond to the parent moiety is through the carbonyl. Non-limiting examples of suitable groups include benzoyl and 1- and 2-naphthoyl.

“Aroylamino” means an aroyl-amino- group in which the aroyl group is as previously described. The bond the to the parent moiety is through the amino group.

“Alkoxy” means an alkyl-O— group in which the alkyl group is as previously described. Non-limiting examples of suitable alkoxy groups include methoxy, ethoxy, isopropoxy, and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkoxy” means an aralkyl-O— group. Non-limiting example of a suitable aralkoxycarbonyl group is benzyloxy. The bond to the parent moiety is through the oxygen atom.

“Alkylamino” means an —NH₂ or —NH₃⁺ group in which one or more of the hydrogen atoms on the nitrogen is replaced by an alkyl group as defined above.

“Dialkylamino” means an NH₂ or —NH₃⁺ group in which two of the hydrogen atoms on the nitrogen is replaced by an alkyl group as defined above.

“Arylamino” means an —NH₂ or —NH₃⁺ group in which one or more of the hydrogen atoms on the nitrogen is replaced by an aryl group as defined above.

“Alkylthio” means an alkyl-S— group in which the alkyl group is as previously described. Non-limiting examples of suitable alkylthio groups include methylthio, ethylthio, i-propylthio and heptylthio. The bond to the parent moiety is through the sulfur.

“Aralkylamino” means an aralkyl-amino in which the aralkylamino is as previously described. The bond to the parent moiety is through the amino group.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Heteroaralkylthio” means a heteroaralkyl-S group in which the heteroaralkyl group is as previously described. The bond to the parent moiety is through the sulfur.

“Thiono” refers to moieties containing the thiocarbonyl group, —C(═S)—. Non-limiting examples include thionoester, —C(═S)—OR, thioamide, —C(═S)—NH2 and the like.

“Alkylthiono” means an alkyl-thiono- group in which the alkyl group is as previously described. The bond to the parent moiety is though the —C(═S)— group.

“Arylthiono” means an aryl-thiono- group in which the alkyl group is as previously described. The bond to the parent moiety is though the —C(═S)— group.

“Aralkylthiono” means an aralkyl-thiono- group in which the alkyl group is as previously described. The bond to the parent moiety is though the —C(═S)— group.

“Cycloalkylthio” means a cycloalkyl-S— group in which the cycloalkyl group is as previously described. Non-limiting example of a suitable cycloalkylthio group is cyclohexylthio. The bond to the parent moiety is through the sulfur.

“Heterocyclothio” means a heterocyclo-S-group in which the heterocyclothio is as previously described. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Alkylsulfinyl” means an alkyl-S(O)— group. Preferred groups are those in which the alkyl group is lower alkyl. The bond to the parent moiety is through the sulfinyl.

“Arylsulfonyl” means an aryl-S(O₂)— group. The bond to the parent moiety is through the sulfonyl.

“Aralkylsulfonyl” means an aralkyl-S(O₂)— group. The bond to the parent moiety is through the sulfonyl.

“Arylsulfinyl” means an aryl-S(O)— group. The bond to the parent moiety is through the sulfinyl.

“Heteroarylsulfonyl” means a heteroaryl-S(O₂)— group. The bond to the parent moiety is through the sulfonyl.

“Heteroarylsulfinyl” means a heteroaryl-S(O)— group. The bond to the parent moiety is through the sulfinyl.

“Heteroarylthio” means an heteroaryl-S— group in which the heteroaryl group is as previously described. The bond to the parent moiety is through the sulfur.

“Amino acids” means a natural amino acid that is bound via covalent amide linkage formed by loss of a molecule of water between the carboxyl group of the amino acid and the amino group of the remainder of the molecule. Examples of amino acids are glycine, alanine, valine, leucine, isoleucine, methionine, proline, phenylalanine, trytophan, serine, threonine, cysteine, tyrosine, asparagines, glutamine, aspartic acid, glutamic acid, lysine, arginine and histidine.

The term “optionally substituted” means optional substitution with the specified groups, radicals or moieties.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound of the present invention effective in treating Typanosoma bruce and thus producing the desired therapeutic effect.

The compounds of formula I form salts which are also within the scope of this invention. Reference to a compound of formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the formula I may be formed, for example, by reacting a compound of formula I with an amount of acid or base, such as an equivalent amount, in a medium such as one in which the salt precipitates or in an aqueous medium followed by lyophilization.

Exemplary acid addition salts include acetates, adipates, alginates, ascorbates, aspartates, benzoates, benzenesulforiates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Compounds of formula I, and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

This invention provides a method of treating Trypanosoma brucei infections, wherein, the compounds of the invention shown herein above, are administered in combination with an additional anti-Trypanosoma brucei agent and/or an anti-Trypanosoma brucei resistance reversing agent. In general, additional anti-Trypanosoma brucei agents and/or an anti-Trypanosoma brucei resistance reversing agents are ones known in the art to treat or prevent Trypanosoma brucei infections, such as, for example, pentamidine isethionate, suramine sodium, melarsoprol, eflornithine and/or are inhibitors of multidrug resistance (e.g. tetandrine).

In general, in combination with, means, additional anti-Trypanosoma brucei agents and/or anti-Trypanosoma brucei resistance reversing agents may be administered prior to, concurrent with, or subsequent to, the administration of a therapeutically effective amount of a compound of the formulas I, II, III, IV and V and the compounds A-U described herein above.

Compounds useful in this invention are exemplified by the following examples, which should not be construed to limit the scope of the disclosure.

In another embodiment, this invention provides a method of treating Trypanosoma brucei infections wherein, the compounds of the invention shown herein above, are administered in combination with an additional anti-Trypanosoma brucei agent and/or an anti-Trypanosoma brucei resistance reversing agent. In general, additional anti-Trypanosoma brucei agents and/or anti-Trypanosoma brucei resistance reversing agents are ones known in the art to treat or prevent Trypanosoma brucei infections, such as, for example, pentamidine isethionate, suramine sodium, melarsoprol, eflornithine and/or are inhibitors of multidrug resistance (e.g. tetandrine).

In general, in combination with, means, additional anti-Trypanosoma brucei agents and/or anti-Trypanosoma brucei resistance reversing agents may be administered prior to, concurrent with, or subsequent to, the administration of a therapeutically effective amount of a compound of the formulas I, II, III, IV and V and the compounds A-U described herein above.

Compounds of the formulas I, II, III, and IV and Compounds of the formula V, methods of making them and examples describing them can be found in U.S. Pat. Nos. 6,011,029 and 6,037,350, respectively, whose entire contents are incorporated herein by reference.

Additional compounds useful in the methods of this invention are exemplified by the following examples 501-512 below, which should not be construed to limit the scope of the disclosure.

Listed below are definitions of various terms used to describe this invention. These definitions apply to the terms as they are used throughout this specification, unless otherwise limited in specific instances either individually or as part of a larger group.

As used herein, the following terms are used as defined below unless otherwise indicated:

MH⁺-represents the molecular ion plus hydrogen of the molecule in the mass spectrum;

BOC-represents-t-BOC-represents-tert-butyloxycarbonyl;

CBZ-represents —C(O)OCH₂C₆H₅ (i.e., benzyloxycarbonyl);

CH₃CN-represents-acetonitrile;

CDCl₃-represents-deuterated chloroform

CH₂Cl₂-represents dichloromethane;

CIMS-represents chemical ionization mass spectroscopy;

DEA-represents diethyl amine;

DMSO-represents dimethyl sulfoxide

DMF-represents N,N-dimethylformamide;

El-represents electron ionization spectroscopy;

Et-represents ethyl;

Et₂O-represents diethyl ether;

EtOAc-represents ethyl acetate;

EtOH-represents ethanol;

HCl-represents hydrochloric acid

IPA-represents isopropanol;

LAH-represents lithium aluminum hydride;

LCMS-represents liquid chromatography mass spectroscopy;

Me-represents methyl;

MeOH-represents methanol;

MgSO₄-represents magnesium sulfate;

MS-represents mass spectroscopy;

FAB-represents FABMS which represents fast atom bombardment mass spectroscopy;

HRMS-represents high resolution mass spectroscopy;

NaOH-represents sodium hydroxide;

Na₂SO₄-represents sodium sulfate;

NaHCO₃-represents sodium bicarbonate;

NH₄OH-represents amonium hydroxide;

NOE-represents nuclear Overhauser effect;

NMR-represents nuclear magnetic resonance spectroscopy;

NMM-represents N-methylmorpholine;

p-TosCI-represents p-toluenesulfonyl chloride;

P₂O₅-represents phosphorous pentoxide;

Pr-represents propyl;

Et₃N-represents TEA which represents triethylamine;

t-BUTYL-represents —C—(CH₃)₃;

TFA-represents trifluoroacetic acid;

THF-represents tetrahydrofuran;

TLC-represents thin layer chromatography;

FPT-represents Farnesyl Protein Transferase

### PREPARATIVE EXAMPLE 1

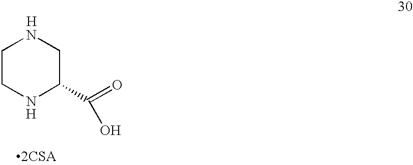

To 2.5 kg of (R)-(−)-camphorsulfonic acid stirring at 60° C. in 1250 ml of distilled water was added a solution of the potassium salt of 2-carboxyl-piperazine (565 gm, 3.35 mol). The mixture was allowed to stir at 95° C. until completely dissolved. The solution was allowed to stand at ambient temperature for 48 hrs. The resulting precipitate was filtered to obtain 1444 gm of damp solid. The solids were then dissolved in 1200 ml of distilled water and heated on a steam bath until all solids dissolved. The hot solution was then set aside to cool slowly for 72 hrs. The crystalline solids were filtered to give 362 gm of pure product (30) as a solid. [α]_{D}=−14.9°

### PREPARATIVE EXAMPLE 2

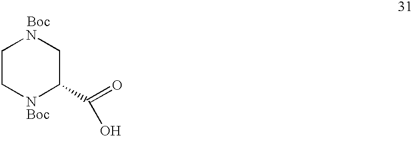

2-R-carboxyl-piperazine-di-(R)-(−)-camphorsulfonic (362 gm, 0.608 mol) was dissolved in 1.4 L of distilled water and 1.4 L of methanol. 75 ml of 50% NaOH was dripped into the stirred reaction mixture to obtain a ˜pH 9.5 solution. To this solution was added di-tert.butyl-dicarbonate (336 gm, 1.54 mol) as a solid. The pH dropped to ˜7.0. The pH of the reaction mixture was maintained at 9.5 with 50% NaOH (total of 175 ml), and the reaction mixture stirred for 2.5 hours to obtain a white precipitate. The reaction mixture was diluted to 9 L with ice/water followed by washing with 2 L of ether. The ether was discarded and the pH of the aqueous layer adjusted to pH 3.0 by the portion wise addition of solid citric acid. The acidified aqueous layer was then extracted with dichloromethane 3×with 2L. The organic layers were combined, dried over sodium sulfate, filtered and evaporated to obtain 201.6 gm of title compound (31) as a solid. FABMS (M+1)=331

### PREPARATIVE EXAMPLE 3

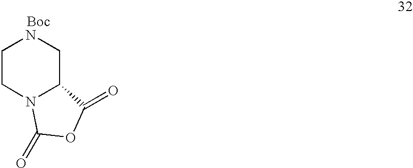

To an ice cold solution N,N-dimethylformamide (49.6 ml) was added, drop wise, thionylchloride (46.7 ml) over a period of 5 minutes in a 5 L round bottom flask under a nitrogen atmosphere. The reaction mixture was allowed to stir for 5 min. and the ice bath removed and the reaction mixture allowed to stir at ambient temperature for 30 min. The reaction mixture was cooled again in an ice bath and a solution of N,N-di-butoxycarbonyl-2-R-carboxyl-piperazine (201.6 gm, 0.61 mmol) in 51.7 ml of pyridine and 1.9 L of acetonitrile was cannulated into the reaction mixture. The reaction mixture was allowed to warm to ambient to obtain a yellowish turbid solution. After stirring at ambient temperature for 18 hours, the reaction mixture was filtered and the filtrate poured into ice water (7L) and then extracted with 4×2 L of ethylacetate, dried over sodium sulfate, filtered and evaporated to dryness under vacuo to obtain 115.6 gm (73%) of the title product (32) as a solid.

### PREPARATIVE EXAMPLE 4

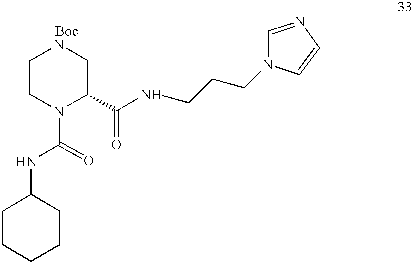

Compound (32) from Preparative Example 3 (0.84 gm, 3.3 mmol) was dissolved in 15 ml of dichloromethane. 1-(3-aminopropyl)imidazole) (0.4 ml, 3.3 mmol) was added drop wise and the reaction stirred at room temperature for 2 hours. After 2 hours 0.2 ml more of 1-(3-aminopropyl)imidazole) was added and the reaction mixture stirred for 2 hours. Cyclohexylisocyanide (0.86 ml, 6.7 mmol) was added drop wise and the reaction mixture stirred for 4 hours. After washing with brine, the dichloromethane was concentrated to dryness and the residue chromatographed on silica gel to obtain the title product (33) (0.9 gm) which was utilized in the next step.

### PREPARATIVE EXAMPLE 5

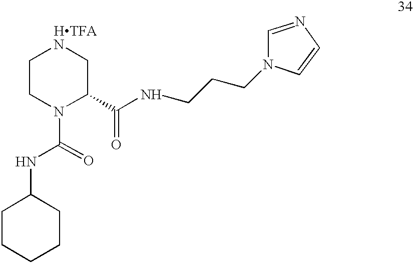

Compound (33) from Preparative Example 4 above, was treated with trifluoroacetic acid for 1 hour. The reaction mixture was evaporated to dryness and was then evaporated from toluene 3 times to obtain the product (34) as an oil (0.9 gm).

### PREPARATIVE EXAMPLE 6

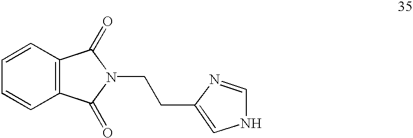

N-Carbethoxyphthalimide (62.8 g, 0.275 mol, 1.1 eq.) was added portion wise over a period of 30 minutes to a stirred solution of histamine dihydrochloride (46.7 g, 0.250 mol, 1.0 eq.) and sodium carbonate (54.3 g, 0.513 mol, 2.05 eq.) in distilled water (1250 ml) at room temperature. The resulting snow-white suspension was stirred vigorously at room temperature for 90 minutes. The solid was filtered off and thoroughly washed with ice-cold distilled water (4×50 ml). The solid was collected and dried under vacuum over P₂O₅ at 60° C. for 12 h to give N^{α}-phthaloylhistamine (35) (59.2 g, 0.245 mol, 98%) in high purity (>95% by ¹H NMR). The solid (35) was used directly without further purification.

¹H NMR (CDCl₃, 200 MHz): 69.50-9.25 (br. s, 1H), 7.80-7.60 (m, 5H), 6.90 (s, 1H), 3.98 (t, 2H, J=7.0 Hz), 3.06 (t, 2H, J=7.0 Hz).

MS (EI): m/e 241 (M⁺, 15%), 160 (34), 94 (B+).

mp 189-191° C. (ethanol, 190 proof).

Reference: Emmett, J. C., Holloway, F. H., Turner, J. L. J. Chem. Soc., Perkin Trans. 1 1979,1341-1344.

### PREPARATIVE EXAMPLE 7

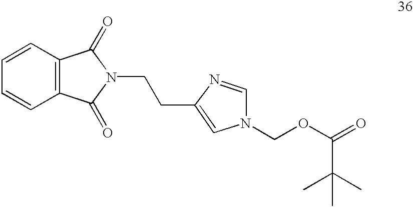

A solution of chloromethyl pivalate (18.5 ml, 0.125 mol, 1.2 eq.) in anhydrous N,N-dimethylformamide (DMF, 100 ml) was added drop wise over a period of one hour to a stirred mixture of Compound (35) (25.0 g, 0.104 mol, 1.0 eq.) and potassium carbonate (17.2 g, 0.125 mol, 1.2 eq.) in anhydrous DMF (500 ml) at 90° C. under a nitrogen atmosphere. The mixture was stirred at 90° C. for 12 h. The volatiles were removed under vacuum at 50° C. The residue was taken up in brine (100 ml) and extracted with ethyl acetate (4×25 ml). The combined organic extracts were dried over Na₂SO₄, filtered, and concentrated under vacuum at 30° C. The residual off-white solid was flash-chromatographed (hexanes:acetone=6:4 v/v) over silica gel to give N^{τ}-pivaloyloxymethyl-N^{α}-phthaloylhistamine 4 (20 g, 0.056 mol, 54%) as a solid (36) of high purity (>95% by ¹H NMR).

¹H NMR (CDCl₃, 200 MHz): δ 7.87-7.76 (m, 2H), 7.75-7.65 (m, 3H), 6.89 (s, 1H), 5.78 (s, 2H), 4.00 (t, 2H, J=7.1 Hz), 2.99 (t, 2H, J=7.1 Hz), 1.14 (s, 9H).

(NOE experiments unequivocally confirmed that the N^{τ} isomer was exclusively isolated.)

MS (FAB+): m/e 356 ([M+H]⁺).

HR-MS (FAB):Calculated for C₁₉H₂₂N₃O₄ ([M+H]⁺): 356.1610. Found: 356.1613.

mp: 126-128° C.

### PREPARATIVE EXAMPLE 8

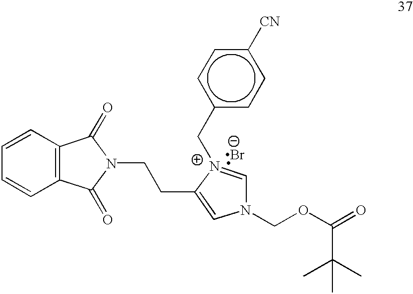

A solution of Compound (36) (10.2 g, 28.7 mmol, 1.0 eq.) and α-bromo-p-tolunitrile (11.4 g, 57.4 mmol, 2.0 eq.) was stirred in anhydrous acetonitrile (150 ml) at 50° C. under a nitrogen atmosphere for 12 h. The resulting snow-white suspension was cooled to room temperature and chilled in a refrigerator at −20° C. for one hour. The precipitate was filtered off and thoroughly washed with ice-cold ethyl acetate (4×50 ml). The solid was collected and dried under vacuum over P₂O₅ at 50° C. for 12 h to give Compound (37) (14.4 g, 26.2 mmol) in 91% yield. The hygroscopic salt (37) was more than 95% pure by ¹H NMR and was used directly without any attempts at purification.

¹H NMR (CD₃OD, 200 MHz): δ 7.80-7.70 (m, 8H), 7.57-7.45 (m, 2H), 6.09 (s, 2H), 5.66 (s, 2H), 3.90 (t, 2H, J=6.4 Hz), 3.05 (t, 2H, J=6.4 Hz), 1.17 (s, 9H).

### PREPARATIVE EXAMPLE 9

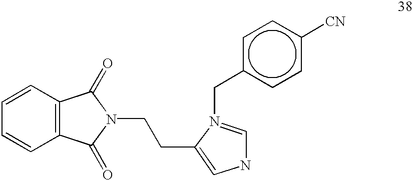

A 7 N solution of ammonia in methanol (75 ml, 0.525 mol, 7.25 eq.) was added drop wise over a period of 75 minutes to a stirred solution of Compound (37) (40 g, 0.073 mol, 1.00 eq.) in anhydrous methanol (1000 ml) at 0° C. under a nitrogen atmosphere. The mixture was slowly (3 h) warmed to ambient temperature and stirred for another 12 h. The volatiles were evaporated under vacuum at 30° C. and the residual solid was flash-chromatographed (CH₂Cl₂:2 N NH₃/MeOH=90:10 v/v) over silica gel to give N^{π}-(4-cyanobenzyl)-N^{α}-phthaloylhistamine Compound (38) (21 g, 0.059 mol, 81%).

¹H NMR (CDCl₃, 200 MHz): δ 7.87-7.70 (m, 4H), 7.68 (s, 1H), 7.63 (d, 2H, J=8.4 Hz), 7.20 (d, 2H, J=8.4 Hz), 6.98 (s, 1H), 5.31 (s, 2H), 3.82 (t, 2H, J=7.6 Hz), 2.81 (t, 2H, J=7.6 Hz).

¹³C NMR (CDCl₃, 300 MHz): δ 168.0, 141.4, 138.2, 134.3, 132.9, 131.8, 128.2, 127.7, 127.2, 123.5, 118.3, 112.2, 48.2, 36.6, 23.0.

MS (FAB+): m/e 357 ([M+H]⁺).

Elemental Analysis:

| | | | |
|---|---|---|---|
| Calculated: | C 70.78 | H 4.53 | N 15.72 |
| Found: | C 70.30 | H 4.52 | N 15.43 |

### PREPARATIVE EXAMPLE 10

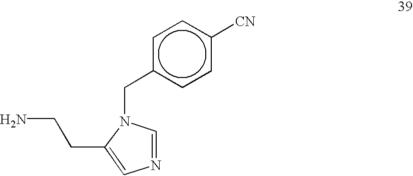

A solution of Compound (38) (21 g, 0.059 mol, 1.0 eq.) and hydrazine monohydrate (15 ml, 0.884 mol, 15.0 eq.) in absolute ethanol (250 ml) was stirred at 50° C. under a nitrogen atmosphere for 12 h. The snow-white suspension was cooled to room temperature and chilled in a refrigerator at −20° C. for one hour. The precipitate (phthalyl hydrazide) was filtered off and thoroughly washed with ice-cold ethanol (190 proof, 500 ml). The filtrates were combined and concentrated under vacuum at 30° C. The residue was subjected to flash column chromatography (CH₂Cl₂:2 N NH₃/MeOH=90:10 v/v) over silica gel to give N^{π}-(4-cyanobenzyl) histamine (39) (11.4 g, 0.050 mol, 85%) as an oil.

¹H NMR (CDCl₃, 200 MHz): δ 7.64 (d, 2H, J=8.3 Hz), 7.53 (s, 1H), 7.12 (d, 2H, J=8.3 Hz), 6.94 (s, 1H), 5.20 (s, 2H), 2.89 (t, 2H, J=6.8 Hz), 2.54 (t, 2H, J=7.6 Hz), 1.37 (br. s, 2H).

¹³C NMR (CDCl₃, 300 MHz): δ 141.9, 137.8, 132.9, 132.8, 129.6, 127.7, 127.0, 118.3, 112.1, 47.9, 40.8, 28.0.

MS (FAB+): m/e 227 ([M+H]⁺).

HR-MS (FAB):

| | |
|---|---|
| Calculated for C₁₃H₁₅N₄ ([M + H]⁺): | 227.1294. |
| Found: | 227.1297. |

### PREPARATIVE EXAMPLE 11

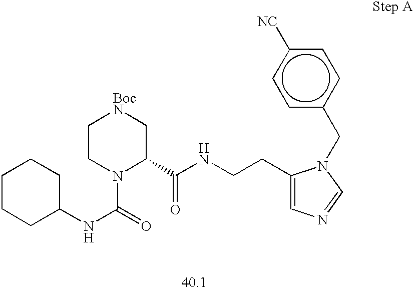

A solution of Compound (39) (1.50 g, 6.63 mmol, 1.0 eq.) in anhydrous dichloromethane (30 ml) was added drop wise over a period of 30 minutes to a stirred solution of anhydride (32) (2.04 g, 7.95 mmol, 1.2 eq.) in anhydrous dichloromethane (30 ml) at room temperature. A stream of nitrogen was bubbled through the solution to expel evolved carbon dioxide. The colorless solution was stirred for one hour amid nitrogen bubbling. Bubbling was terminated and cyclohexyl isocyanate (1.75 ml, 13.26 mmol, 2.0 eq.) was added drop wise over a period of 5 minutes. The brown solution was stirred at room temperature for one hour to give the Boc protected piperazine intermediate (40.1) (confirmed by ¹H NMR) which was reacted further without purification in Step B below.

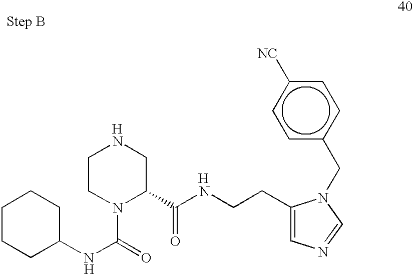

The solution mixture containing Compound (40.1) from Step A, above, was concentrated under vacuum at 30° C. The residue was then taken up in a mixture of trifluoroacetic acid (30 ml) and anhydrous dichloromethane (30 ml) and stirred at ambient temperature under a nitrogen atmosphere for 24 h. The mixture was concentrated under vacuum at 30° C. The residual light-brown oil was taken up in 1N aqueous NaOH solution (100 ml) and extracted with dichloromethane (4×25 ml). The combined organic extracts were washed with brine (25 ml), dried over Na₂SO₄, filtered, and concentrated under vacuum at 30° C. The resulting oil was flash-chromatographed (CH₂Cl₂:2 N NH₃/MeOH=90:10 v/v) over silica gel to give Compound (40) (1.34 g, 2.95 mmol, 45%) as a foam.

¹H NMR (CDCl₃, 200 MHz): δ 7.97 (br. s, 1H), 7.65 (d, 2H, J=8.3 Hz), 7.52 (s, 1H), 7.14 (d, 2H, J=8.3 Hz), 6.90 (s, 1H), 5.40 (d, 2H, J=6.8 Hz), 5.21 (s, 2H), 4.28 (d, 1H, J=2.6 Hz), H 3.9-1.0 (m, 21H).

MS (FAB+): m/e 464 ([M+H]⁺).

HR-MS (FAB):

| | |
|---|---|
| Calculated for C₂₅H₃₄N₇O₂ ([M + H]⁺): | 464.2774. |
| Found: | 464.2768. |

### PREPARATIVE EXAMPLE 12

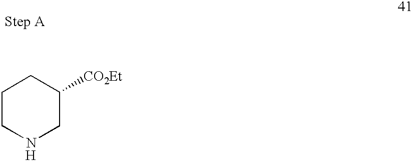

Ethyl nipecotate (70.2 g, 0.446 mmol) and D-tartaric acid (67.0 g, 1.0 eq.) were dissolved in hot 95% EtOH (350 mL). The resulting solution was cooled to room temperature, filtered, and the crystals washed with ice-cold 95% EtOH. The product was then recrystallized from 95% EtOH (550 mL) to give ethyl (S)-nipecotate D-tartrate (38.5 g, 56% yield). The salt (38.5 g) was dissolved in water (300 mL), cooled to 0° C., and 3M NaOH was added until the pH was 9-10. The resulting solution was extracted with CH₂Cl₂ (5×100 mL) and the combined organics dried over Na₂SO₄, filtered, and concentrated under reduced pressure to give Compound (41) as an oil (19 g, 89% yield). CIMS: MH⁺=158.

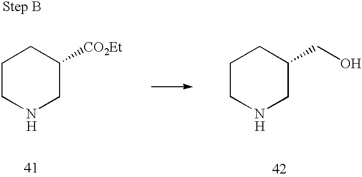

To a solution of Compound (41) from Step A (18.5 g, 0.125 mol) in THF (250 mL) was added LAH (118 mL, 1.0 M in Et₂O, 1.0 eq.) at 0° C. over 20 minutes. The resulting solution was warmed to room temperature, then to reflux and stirred 2 hours. The reaction mixture was recooled to room temperature and quenched by the slow addition of saturated Na₂SO₄. The resulting slurry was dried by the addition of Na₂SO₄, diluted with EtOAc (250 mL), filtered through a plug of Celite, and concentrated to give Compound (42) as an oil (13.7 g 100% crude yield) which was used without purification. CIMS: MH⁺=116; [α]²⁰_{D}=−8.4° (5.0 mg in 2.0 mL MeOH).

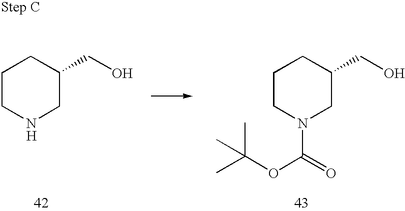

Compound (42) from Step B above (13.6, 0.104 mmol) was dissolved in MeOH (100 mL) and H₂O (100 mL). Di-tert-butyl dicarbonate (27.2 g, 1.2 eq.) was added portion wise keeping the pH>10.5 by the addition of 50% NaOH. The resulting solution was stirred at room temperature for 2.5 hours. The reaction mixture was concentrated in vacuo, diluted with H₂O (300 mL) and extracted with CH₂Cl₂ (3×150 mL). The combined organics were dried over Na₂SO₄, filtered, and concentrated under reduced pressure. The crude product was purified by flash chromatography eluting with 50% EtOH in hexane (12.1 g, 48% yield). CMS: 216 (MH⁺=100); [α]²⁰_{D}=+15.20 (5.0 mg in 2.0 mL MeOH).

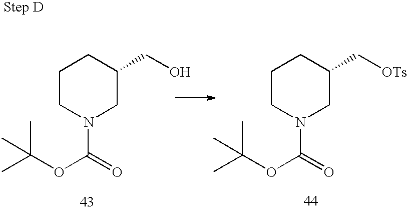

pTosCl (12.8 g, 1.2 eq.) was added portion wise to Compound (43) from Step C (12.0 g, 55.7 mmol) in pyridine (120 mL) at 0° C. The resulting solution was stirred 0° C. overnight. The reaction mixture was diluted with EtOAc (300 mL) and washed with cold 3N HCl (5×300 mL), saturated NaHCO₃ (2×150 mL), H₂O (1×100 mL), brine (1×100 mL), dried over Na₂SO₄, filtered, and concentrated in vacuo to give Compound (44) as an oil which was used without purification (100% crude yield). FABMS: MH⁺=370.

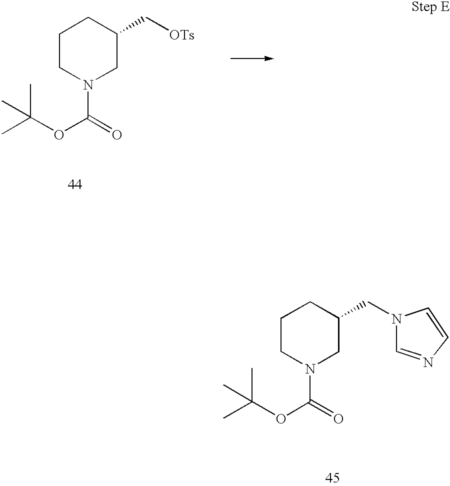

Compound (44) from Step D above (21.0 g, 5.7 mmol) in DMF (300 mL) was treated with sodium imidazole (8.37 g, 1.5 eq.). The resulting solution was stirred, heated to 60° C., and stirred 2 hours. The reaction mixture was cooled to room temperature and concentrated in vacuo. The residue was diluted with H₂O (300 mL) and extracted with CH₂Cl₂ (3×150 mL). The combined organics were dried over Na₂SO₄, filtered, and concentrated under reduced pressure. The crude product was purified by flash chromatography using a 7% MeOH in CH₂Cl₂ solution as eluent to give Compound (45) as a solid (7.25 g, 49% yield). FABMS: MH⁺=266; [α]²⁰_{D}=+8.0° (5.0 mg in 2.0 mL MeOH).

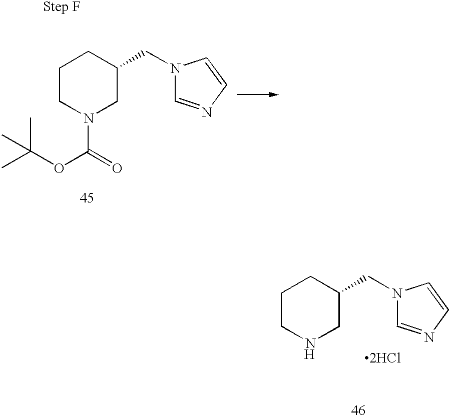

The title compound (45) from Step E (5.50 g, 20.7 mmol) was stirred at room temperature in 4M HCl/dioxane (50 mL) overnight. The resulting solution was concentrated in vacuo and triturated with Et₂O to give Compound (46) as a solid. CIMS: MH⁺=166.

### PREPARATIVE EXAMPLE 13

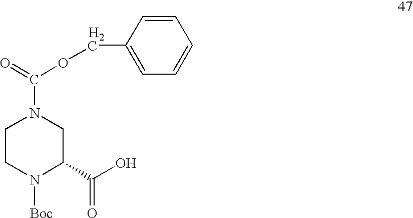

2-Carboxy-piperazine-dicamphorsulfonic acid salt (30)(Preparative Example 1) (17.85 gm, 30 mmole) was dissolved in 180 ml of distilled water. Dioxane (180 mL) was added and the pH adjusted to 11.0 with 50% NaOH. The reaction mixture was cooled to 0-5° C. in an ice-MeOH bath and a solution of benzyl-chloroformate (4.28 mL, 30 mmol) in 80 mL of dioxane was added over a period of 30-45 minutes while stirring at 0-5° C. and keeping the pH at 10.5 to 11.0 with 50% NaOH. After the addition was complete, stirring was continued for 1 hr. The reaction mixture was then evaporated to dryness (to get rid of the dioxane for extraction). The residue was dissolved in 180 mL of distilled water and the pH adjusted slowly to 4.0 with 1N HCl. The aqueous solution was washed with 3×180 mL of ethyl acetate (The ethyl acetate was dried over MgSO₄, filtered, and evaporated to obtain N,N-di-CBZ-2-carboxy-piperazine and saved). The pH of the aqueous layer, which contains the desired product, was adjusted to 10.5 to 11.0 with 50% NaOH and solid di-tert-butyl-dicarbonate (7.86 gm, 36 mmol) was added and the mixture was stirred while keeping the pH at 10.5 to 11.0 with 50% NaOH. After 1 hr. the pH stabilized. When the reaction was complete, the reaction mixture was washed with 2×180 mL of Et₂O. The aqueous layer was cooled in an ice bath and the pH was adjusted to 2.0 with 1N HCl (slowly). The reaction mixture was extracted with 3×200 mL of ethyl acetate, dried over MgSO₄, filtered and concentrated to obtain 9.68 gm (88%) of pure product (47) as a solid.

### PREPARATIVE EXAMPLE 14

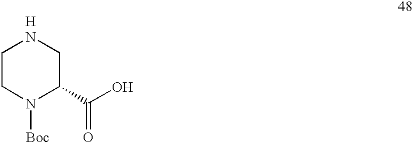

Compound (47) from Preparative Example 13 (9.6 gm, 26.3 mmol) was dissolved in 100 mL of absolute ethanol in a hydrogenation vessel. The vessel was flushed with nitrogen and 3 gm of 10% Pd/C (50% by weight with water) was added. The mixture was hydrogenated at 55 psi of H₂ for 18 hours. After 18 hrs, the reaction mixture had a precipitate. The TLC was checked (30% MeOH/NH₃/CH₂Cl₂). The reaction mixture was filtered on a pad of Celite, and the pad washed with EtOH followed by distilled water. The filtrate was evaporated to ˜⅓ the volume (to get rid of the EtOH) and 200 mL of distilled water was added. The aqueous layer was extracted with ethyl acetate three times (the ethyl acetate layer containing pure N,N-Di-Boc-2-carboxy-piperazine was saved). The water layer was evaporated to dryness and evaporated from methanol two times to obtain 3.98 gm of pure product (48).

### PREPARATIVE EXAMPLE 15

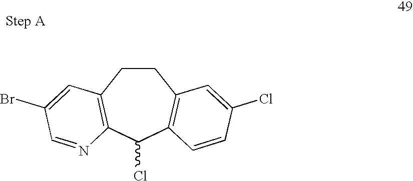

The tricyclic alcohol (Preparative Example 40 in WO 95/10516)(5.6 gm, 17.33 mmol) was dissolved in 56 ml of dichloromethane and 2.46 ml of thionyl chloride was added while stirring under a dry nitrogen atmosphere. After 5 hours the TLC was checked (by adding an aliquot of the reaction mixture to 1N NaOH and shaking with dichloromethane and checking the dichloromethane layer by TLC using 50% EtOAc/Hexanes as the eluent). The mixture was evaporated to give a gum, which was evaporated, twice from dry toluene and once from dichloro-methane to give the 11-chloro derivative (49) as a solid, which was used without further purification.

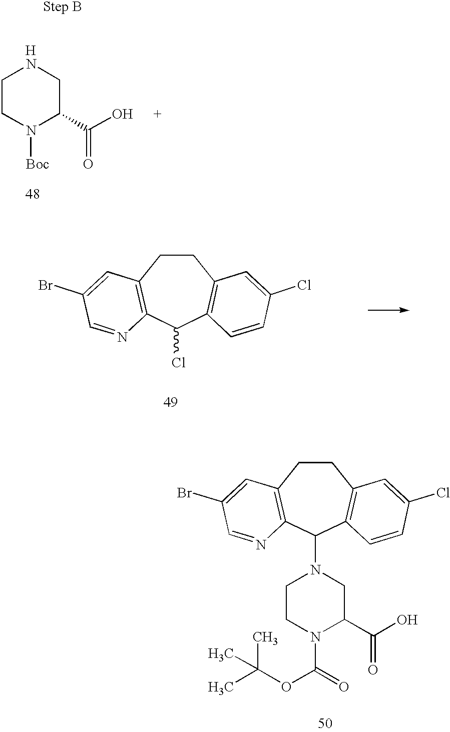

Compound (49) from Step A above, was dissolved in 100 ml of dry DMF, and Compound (48) from Preparative Example 14 (3.98 gm) was added followed by 12.11 ml of triethylamine. The mixture was stirred at ambient temperature under a nitrogen atmosphere. After 24 hours the DMF was evaporated and the residue dissolved in 200 ml of ethyl acetate and washed with brine. The brine layer was washed with ethyl acetate two more times and the ethyl acetate layers combined, dried over magnesium sulfate, filtered, and evaporated to give a solid. The solid was chromatographed on a 1½″×14″ column of silica gel eluting with 2L of 0.4% 7N MeOH/NH₃:CH₂Cl₂, 6L of 0.5% 7N MeOH/—NH₃:CH₂Cl₂, 2L of 0.65% 7N MeOH/NH₃:CH₂Cl₂, 2L of 0.8% 7N MeOH/NH₃:CH₂Cl₂, 4L of 1% 7N MeOH/NH₃:CH₂Cl₂, 2L of 3% 2N MeOH/NH₃:CH₂Cl₂, 2L of 5% 2N MeOH/NH₃:CH₂Cl₂, 2L of 10% 2N MeOH/NH₃:CH₂Cl₂, 2L of 15% 2N MeOH/NH₃:CH₂Cl₂, 4L of 20% 2N MeOH/NH₃:CH₂Cl₂ to obtain 4.63 gm of final product (50).

### PREPARATIVE EXAMPLE 16

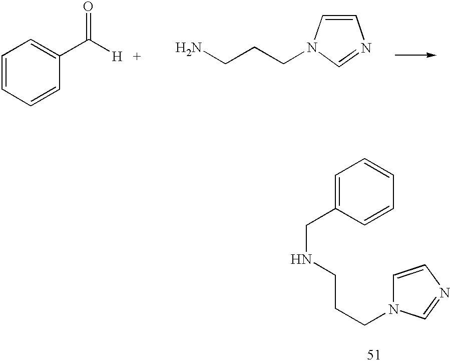

A mixture of 1-(3-aminopropyl)imidazole (37.1 g, 297 mmol), benzaldehyde (30 g, 283 mmol), 3 Å molecular sieves (50 g), sodium acetate (24.1 g, 283 mmol) and anhydrous methanol (700 mL) was stirred at room temperature under N₂ overnight. The mixture was cooled to 0° C. and sodium borohydride (10.9 g, 288 mmol) was added portion wise over 1 hour. The mixture was stirred at room temperature for 3 hours. The mixture was filtered through celite, washed with methanol, and concentrated in vacuo to give a residue, which was diluted with dichloromethane and washed with 10% aqueous sodium hydroxide. The organic phases were washed with brine, dried over anhydrous magnesium sulfate, filtered and concentrated in vacuo to give the title compound as an oil (56.3 g, 92%, MH⁺=216).

### PREPARATIVE EXAMPLE 17

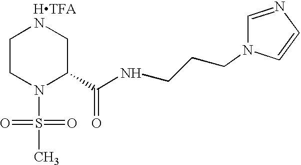

In essentially the same manner as Preparative Examples 4 and 5, except substituting methanesulfonyl chloride for cyclohexylisocyanide, Compound (52) was prepared.

### PREPARATIVE EXAMPLE 18

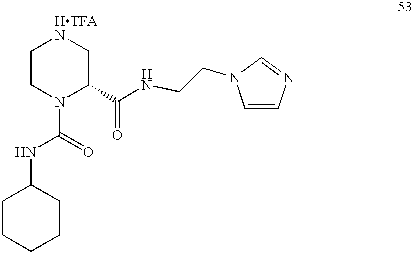

In essentially the same manner as Preparative Examples 4 and 5, except substituting 1-(2-aminoethyl)imidazole) for 1-(3-aminopropyl)imidazole), Compound (53) was prepared.

### PREPARATIVE EXAMPLE 19

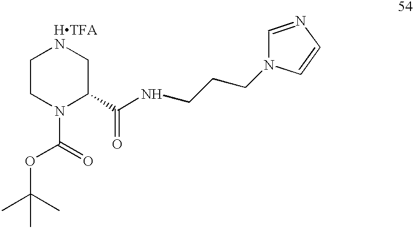

Compound (32) from Preparative Example 3 was treated with 1-(3-aminopropyl)imidazole in essentially the same manner as Preparative Example 4 to afford the t-Boc derivative, which was further reacted in a similar manner as in Preparative Example 5 to afford Compound (54).

### PREPARATIVE EXAMPLE 20

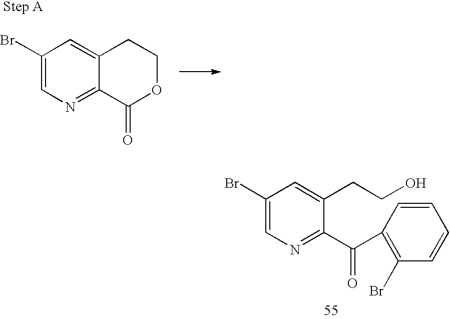

To a solution of 1,2-iodobromobenzene (11.5 mL) in 840 ml of tetrahydrofuran, cooled to −78° C. and under a dry nitrogen atmosphere was added 45.5 mL of a 2M ether solution of isopropylmagnesiumchloride. After stirring for 30 minutes, a solution of 3-bromo-5,6-dihydro-2H-pyrano[3,4-b]pyridin-8-one (14 gm, 61.67 mmol) in 100 mL of tetrahydrofuran was added and the reaction mixture stirred for 1 hr. The reaction was then poured into a solution of saturated ammonium chloride and the resulting mixture extracted three times with ethyl acetate. The combined ethyl acetate layers were then dried over magnesium sulfate, filtered, and evaporated to give a solid. The solid was crystallized from ethylacetate/hexanes to obtain 21.82 g of compound (55) FABMS M⁺1=384.

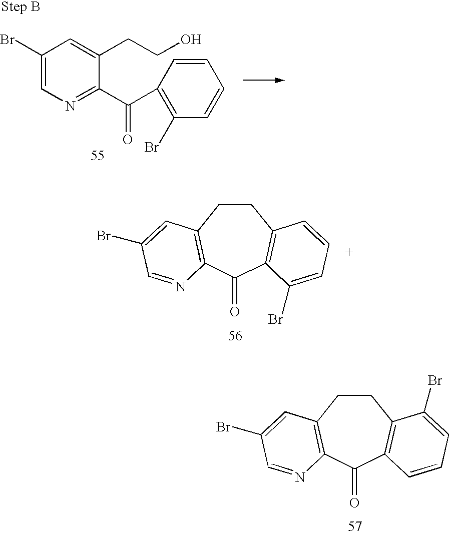

Compound (55) from Step A (15 gm, 39 mmol) was mixed with 67 gm of aluminum chloride and the mixture was heated to 160° C. in a round bottom flask for 2 hours. While cooling the reaction in an ice bath, ice, followed by 500 ml of water was added. Sodium hydroxide (50%) was added to adjust the pH to 12. The resulting solution was extracted three times with ethyl acetate to obtain 9.75 gm of a crude mixture of products which was further purified by chromatography to obtain 1.25 gm of compound (56) FABMS M⁺1=365.

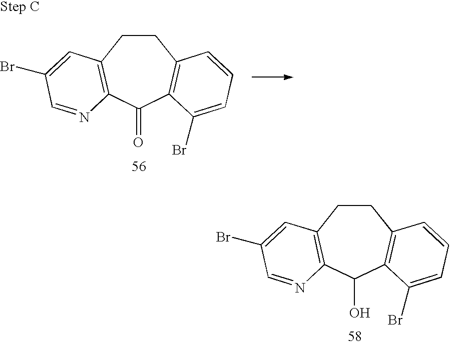

To a solution of Compound (56) from Step B (1.2 gm, 3.3 mmol) in 20 ml of methanol at room temperature was added, portion wise, sodium borohydride (0.29 gm, 5 mmol). After 1 hour, 30 ml of 1N hydrochloric acid was added and the mixture stirred for 5 minutes. To the reaction was added, 1N sodium hydroxide (50 ml) and the resulting mixture extracted with dichloromethane to obtain 1.18 gm of title compound which was used directly in the next step without further purification FABMS M⁺1=367.

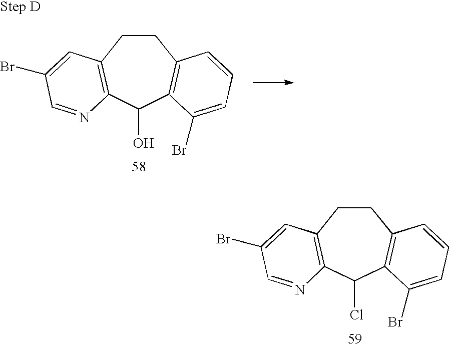

Compound (58) from Step C, was treated in a manner similar to that described in Preparative Example 15, Step A, to obtain compound (59)

### PREPARATIVE EXAMPLE 21

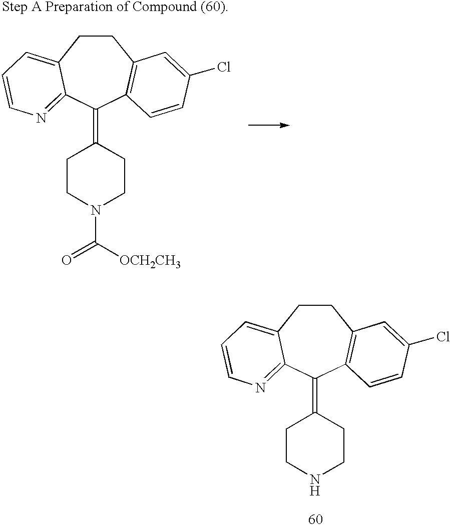

Loratadine® (448 g, 1.17 mol) was refluxed in 2 L of 70% aqueous HCl (1.4 L conc.HCl in 600 ml H₂O) for 12 h. The reaction mixture was then cooled and poured into ice. It was then basified with 950 mL of 50% NaOH followed by extraction with CH₂Cl₂ (1×4L, and 2×2.5L). The organic phase was washed with brine, dried over Na₂SO₄ and MgSO₄ and then filtered. All the volatiles were then removed to give 368 g of the title compound (60). MH⁺=311

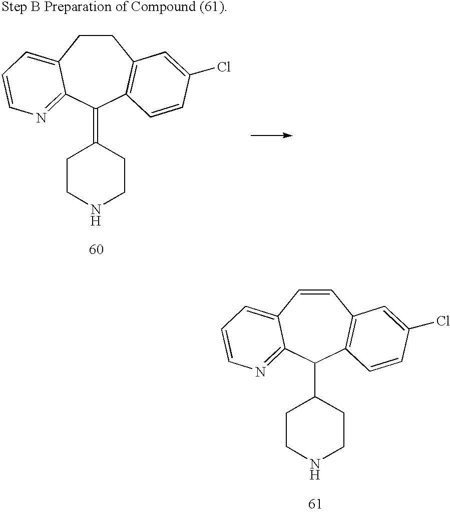

To the title compound from Preparative Example 21, Step A (363 g, 1.17 mol) was added trifuromethane sulfonic acid (1.8 Kg) under N₂. The reaction mixture was refluxed at 170° C. The progress of the reaction was monitored by ¹H NMR. After 4 days the reaction was only 63% complete. After 8 days the reaction was found to be 80% complete according to ¹H NMR; thus another 130 mL of CF₃SO₃H was added and refluxing continued for another 24 h. It was then poured into ice and basified with 800 mL of NaOH (50%) and extracted twice with CH₂Cl₂ (1×8L then 1×7L). The organic phase was combined, washed with H₂O and filtered through celite. It was then dried over MgSO₄ and Na₂SO₄ and again filtered through celite. The filtrate was concentrated to give a semi-solid that was pre-adsorbed on 600 g of silica gel and then chromatographed on 2.3 Kg of silica gel eluting first with 5% CH₃OH—CH₂Cl₂ (saturated with ammonia) and then with 10% CH₃OH—CH₂Cl₂ (saturated with ammonia) to give 102 g of the title compound (61) as a solid. mp=73-75; MS (FAB) m/z 483 (MH⁺).

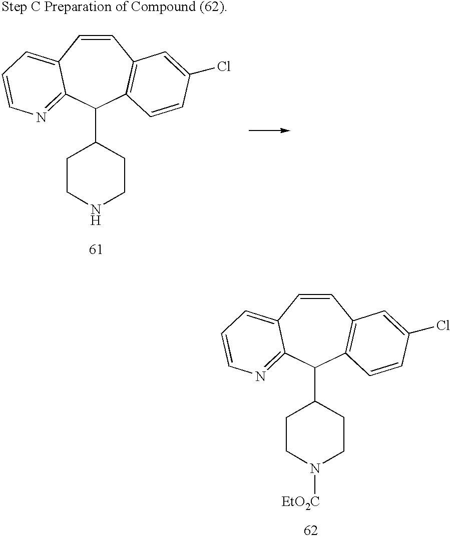

To a solution of the title compound of Preparative Example 21, Step B (145 g) in 1L of CH₂Cl₂ at 0° C. was added ethylchloroformate (55 mL), dropwise. The reaction mixture was stirred at room temperature overnight. It was further diluted with 1L CH₂Cl₂ and stirred with 2L of dilute NaHCO₃, pH˜7-8. The organic layer was separated and dried over MgSO₄ and Na₂SO₄, filtered and concentrated to afford 174 g of a gum. The crude compound was purified by silica gel column chromatography, eluting with 20-60% ethyl acetate-hexane to afford the title compound (62). MS (FAB) m/z 383 (MH⁺).

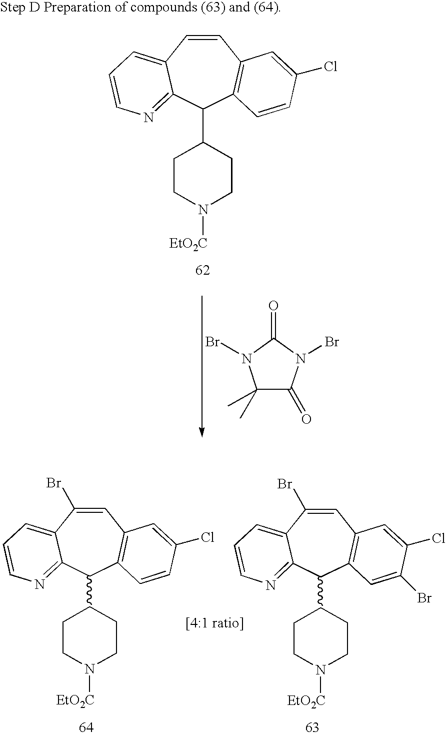

The title compound of Preparative Example 21, Step C (251 g, 0.65 mol) was dissolved in 1.65 L of CH₂Cl₂ and dibromo-dimethylhydantoin (132 g, 0.462 mol) was then added. The solution was stirred until the system was homogeneous. The solution was cooled to 0° C. under N₂ atmosphere and 174 mL of CF₃SO₃H was added over 37 min. while keeping temperatures between ⁻1° C. to ⁺1° C. The reaction mixture was stirred for 3 h, cooled to ⁻10° C. and basified with 50% NaOH (170 mL), keeping the temperature below ⁺1° C. The aqueous phase was extracted with CH₂Cl₂ and then dried over MgSO₄ and concentrated to give 354 g of a foam that was chromatographed on silica gel eluting with 10-50% of ethyl acetate-hexanes gradient to give 50 g of compound (63) (14% yield) and 147 grams of the desired title compound (64) (49% yield). Compound (64) MS m/z (rel intens) 462 (MH⁺); Compound (63) MS m/z (rel intens) 542 (MH⁺).

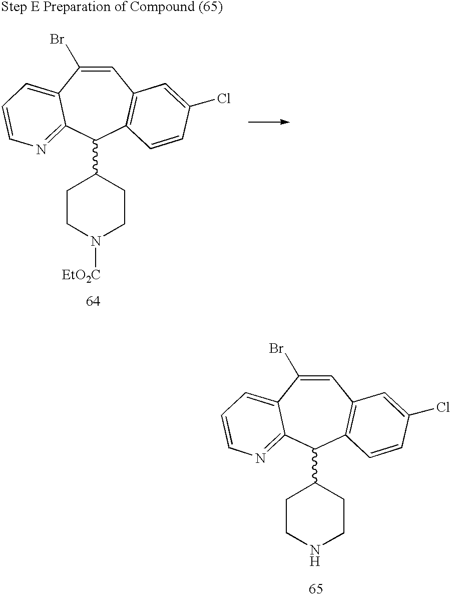

Compound 64 from Preparative example 21, Step D (10.0 g, 21.7 mmol) was refluxed in 2 L of 70% aqueous HCl (1.4 L conc. HCl in 600 ml H₂O) for 12 h. The reaction mixture was then cooled and poured into ice. It was then basified with 950 mL of 50% NaOH followed by extraction with CH₂Cl₂ (1×4L, and 2×2.5L). The organic phase was washed with brine, dried over Na₂SO₄ and MgSO₄, and then filtered. All the volatiles were then removed to give the title compound (65). MH⁺=389.

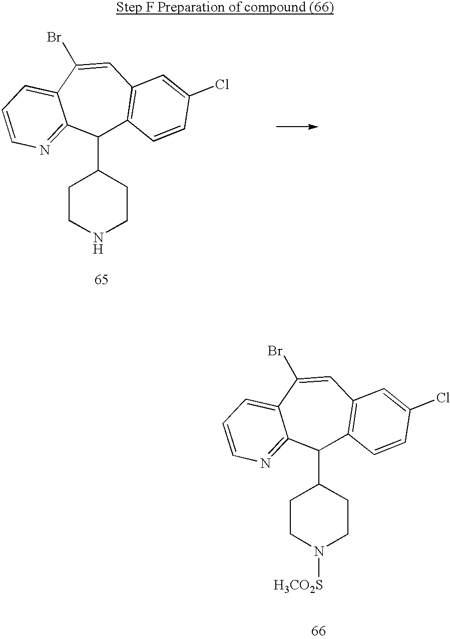

To a solution of the amine product (65) from Preparative Example 21, Step E (20.0 g) in CH₂Cl₂ (100 ml) was added triethyl amine (14.4 ml). Slowly, methane sulfonyl chloride (6.0 ml) was added and the mixture stirred over night at room temperature. To the reaction was added saturated sodium bicarbonate and then it was extracted with CH₂Cl₂. The organic layer was dried over magnesium sulfate, filtered and concentrated to dryness. The crude product mixture was purified with column chromatagraphy, eluting with 1% MeOH/NH₃—CH₂Cl₂, to afford the desired compound (66). MS 469 (MH⁺).

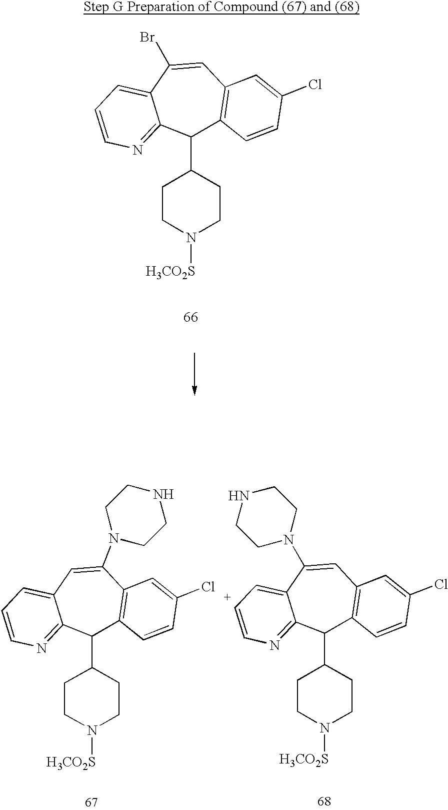

To the title compound from Preparative Example 21, Step F (21.25 g, 45.3 mmol, 1 eq.) in 400 mL of THF was added 19.5 g (266.6 mmol, 5 eq.) of piperazine. The reactants stirred at room temperature until everything was in solution. To this mixture was added potassium t-butoxide 12.7 g (113.3 mmol, 2.5 eq.) in one portion. The reaction mixture was stirred at room temperature for 2 h. All of the THF was removed by rotary evaporation and the resulting crude product was extracted with EtoAc and washed with water. The organic layer was dried over Na₂SO₄ and concentrated to give a mixture of title compounds (67) and (68).

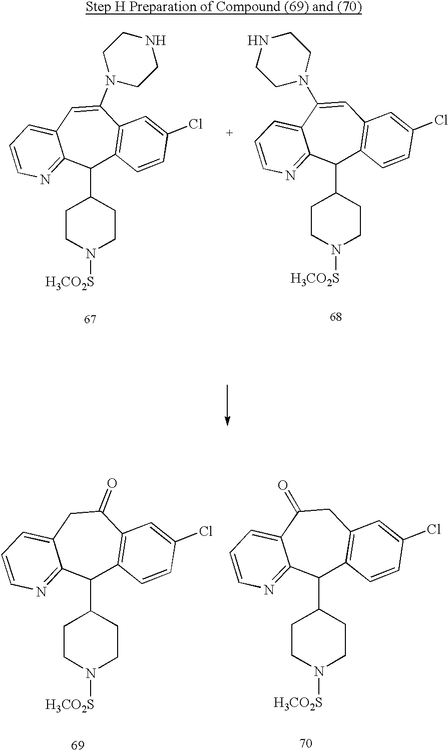

The product from Preparative Example 21, Step G (22.25 g) was dissolved in 150 mL of conc. HCl and stirred for 16 h. The reaction mixture was poured into ice, basified with conc. NH₄OH and then extracted with CH₂Cl₂ to give a mixture of compounds (69) and (70).

### PREPARATIVE EXAMPLE 22

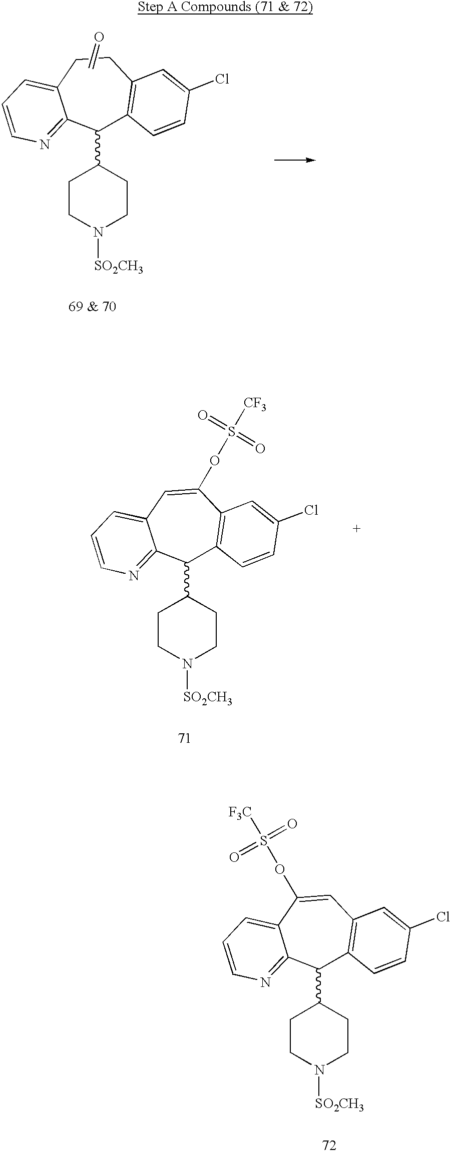

To a mixture of piperazinyl compounds 69 & 70, from Preparative Example 21, Step H in THF (150 mL) at −78° C. was added LDA 4.05 mL (1.1 eq.) and the solution stirred for 1.5 h. The mixture was warmed to −20° C. and then N-phenyl trifluoromethane sulfonimide 2.94 g (1.1 eq.) was added. The reaction stirred over night at room temperature. After all of the THF was removed by rotatary evaporation, the resulting crude product was purified by Biotage column chromatography eluting with 50% EtoAc—Hex, (normal phase) to yield 1.94 g of the desired compound (71).

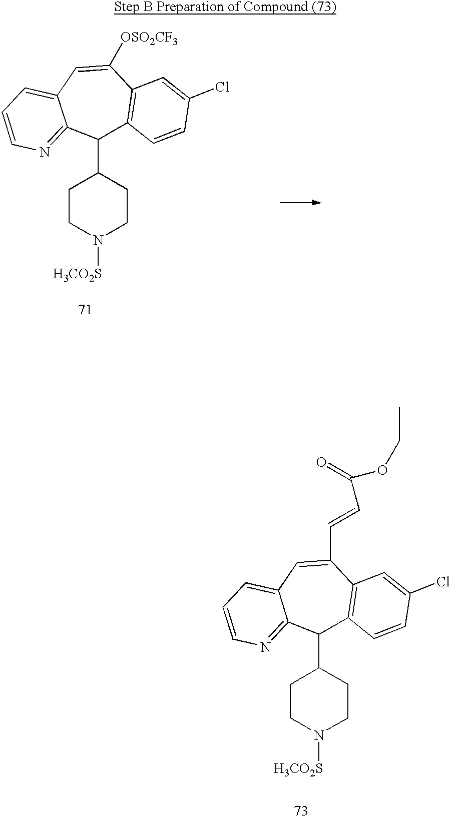

Compound (71) from Preparative example 22, Step A above, was dissolved in DMF. Successively, Et₃N (29 eq.), Ethyl acrylate (5.4 eq.), K₂CO₃ (5 eq.), Bu₄NBr (2 eq.) and Palladuim (11) acetate (0.13 eq.) were added. The mixture was stirred and heated to 100° C. for 4 h. After cooling, the mixture was concentrated and the residue was taken up in CH₂Cl₂ and extracted with CH₂Cl₂/H₂O. The organic layer was dried over Na₂SO₄ then concentrated and the residue purified by Biotage chromatography using 1% MeOH/NH₃—CH₂Cl₂ to afford the title compound (73).

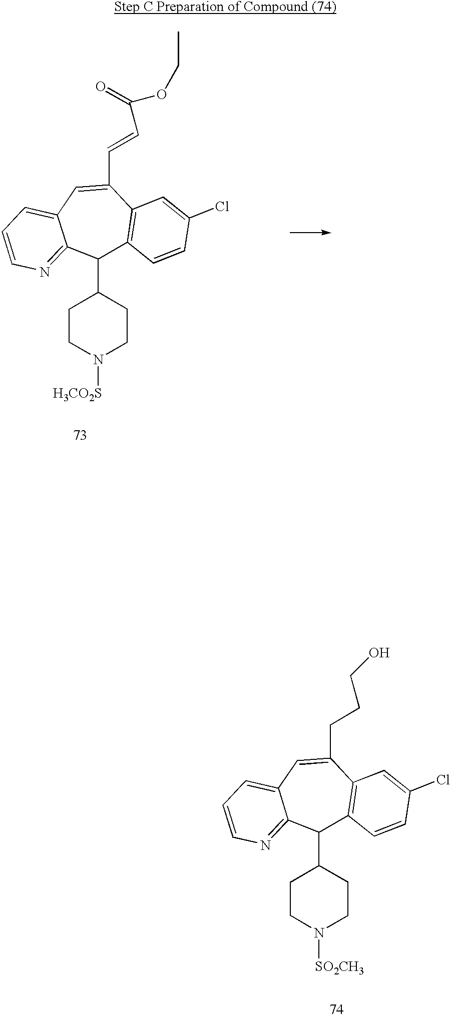

Compound (73) was dissolved in EtOH, cooled in an ice bath and reacted with NaBH₄ (15 eq.) for 3 min. Then CuCl (2 eq) was added and the reaction mixture was stirred for 6 h. at room temperature. The mixture was filtered, concentrated and extracted with CH₂Cl₂. The organic layer was washed with sat. NaHCO₃, dried over Na₂SO₄ and concentrated to yield the title compound (74).

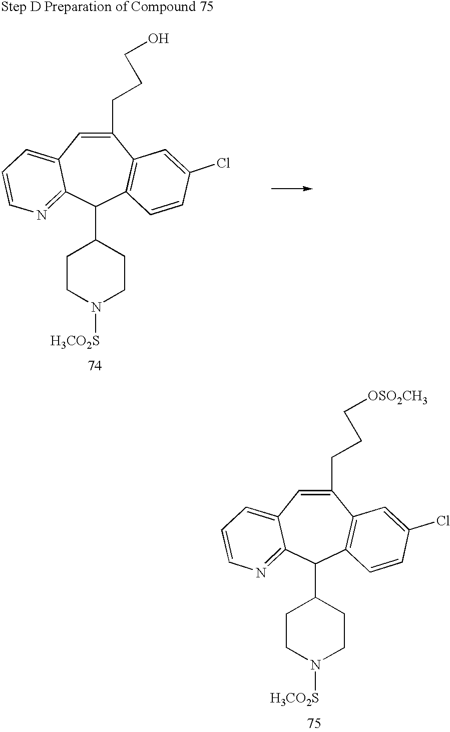

To a solution of Compound 74 (1.24 g) in CH₂Cl₂ (100 ml) was added triethyl amine (1.1 ml). Slowly, methane sulfonyl chloride (0.3 ml) was added and the mixture stirred over night at room temperature. To the reaction was added saturated sodium bicarbonate and then it was extracted with CH₂Cl₂. The organic layer was dried over magnesium sulfate, filtered and concentrated to dryness. The crude product mixture was purified with column chromatography, eluting with 80% EtOAc—Hex, to afford the title compound (75).

### PREPARATIVE EXAMPLE 23

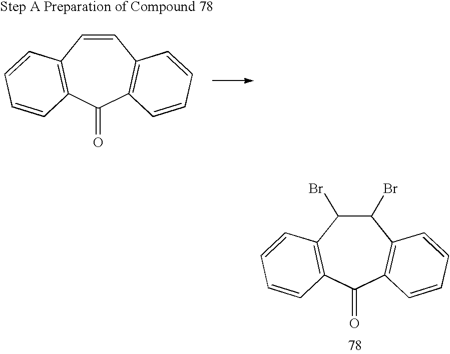

To a stirred solution of bromine (33.0 g, 210 mmol) in CCl₄ (100 ml) was added a solution of dibenzosuberenone (37.0 g, 179 mmol) in CCl₄ (200 ml) at room temperature. The resulting solution was stirred at room temperature for 1.5 hours. The crystals were collected by filtration to give the product (78) (60.12 g, 92% yield, M+H=367).

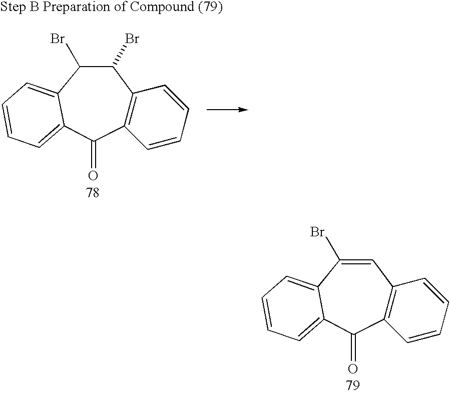

A solution of the di-bromo compound (78) (10.0 g, 27.3 mmol) and NaOH (3.0 g, 82.0 mmol) in MeOH (200 ml) was stirred and heated to reflux for 1.5 hours. The reaction mixture was then cooled to room temperature and stirred overnight. The mixture was evaporated to dryness and then extracted with CH₂Cl₂—H₂O. The combined organic layer was dried over MgSO₄, filtered and evaporated to dryness to give a solid (79) (8.0 g, 100% yield, M=285)

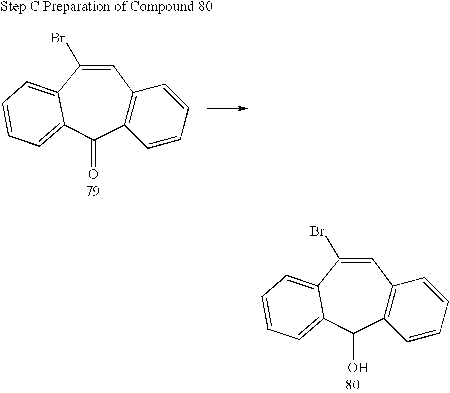

To a stirred solution of the mono-bromo compound (79) from step B (3.9 g, 13.7 mmol) in MeOH (200 ml) under nitrogen and at 0° C. was added NaBH₄ (0.7552 g, 20.0 mmol). The resulting solution was stirred at 0° C. for 3 hours, then evaporated, followed by extraction with CH₂Cl₂—H₂O. The combined organic layer was dried over MgSO₄, filtered, and evaporated to dryness to give a solid (80) (4.1 g, 100%, M=287).

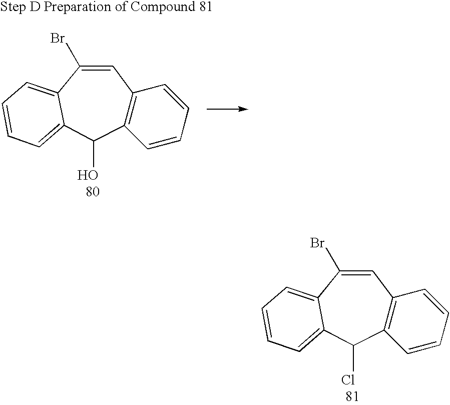

To a stirred solution of alcohol (80) from Preparative Example 3, Step C (3.9 g, 13.6 mmol) in CH₂Cl₂ (100 mL) under nitrogen at −20° C. was added thionyl chloride (13.9 mL, 1.0 mmol). The resulting solution was stirred at room temperature overnight and then evaporated to dryness. The crude mixture was diluted with toluene (50 mL), followed by the addition of more SOCl₂ (13.9 mL) at room temperature. The resulting solution was heated to reflux for 2 hours until the reaction went to completion. The reaction mixture was then cooled to room temperature and concentrated to dryness to give a solid (81) (5.0 g, 100% yield, M-BrCl=191)

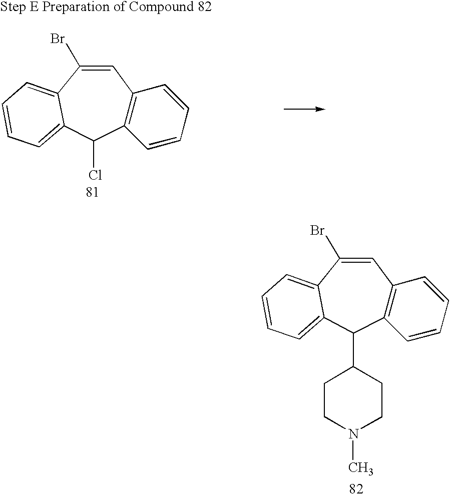

To a suspension of Mg (3.63 g) in anhydrous THF (95 mL) under nitrogen and at room temperature was added 4-chloro-1-methyl piperidine (3 mL, 10% of the total amount) and one small crystal of iodine. The resulting solution was heated to reflux, followed by the addition of iodomethane (0.5 mL) and the remainder of the 4-chloro-1-methyl piperidine (27 mL). The reaction was stirred for one hour and then concentrated to dryness to give the crude Grignard reagent (0.8M).

To a stirred solution of the chloro compound (81) from Preparative Example 3, Step D (3.9 g, 13.7 mmol) in anhydrous THF (40 mL) under nitrogen at 0° C. was added dropwise the Grignard reagent (obtained above) (0.8M, 19.5 mL, 15.6 mmol). The resulting solution was stirred at 0° C. for 1 hour. The reaction mixture was quenched with 100 mL of 15% aq. NH₄Cl solution, followed by the extraction with EtOAc-H₂O. The combined organic layer was dried over MgSO₄, filtered and evaporated to dryness to give the product (82) (5.29 g, 100% yield, MH⁺=368).

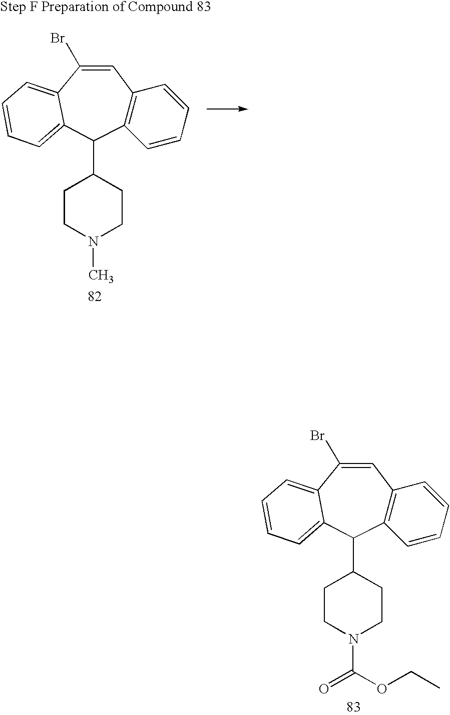

To a stirred solution of Compound (82) from Step E above (5.0 g, 13.6 mmol) in toluene (100 mL) under a nitrogen atmosphere, was added triethylamine (5.7 mL, 40.7 mmol). The resulting solution was heated to refux, then dropwise ethyl chloroformate (13.0 mL, 136.7 mmol) was added. The solution continued to stir at the reflux temperature for 2 hours. The reaction was then stirred at room temperature overnight, followed by extraction with an EtOAc-1N NaOH solution. The combined organic layer was dried over MgSO₄, filtered, concentrated to dryness and the crude product purified by column chromatography on normal phase silica gel, eluting with 10% EtOAc/90% Hexane to give compound (83).

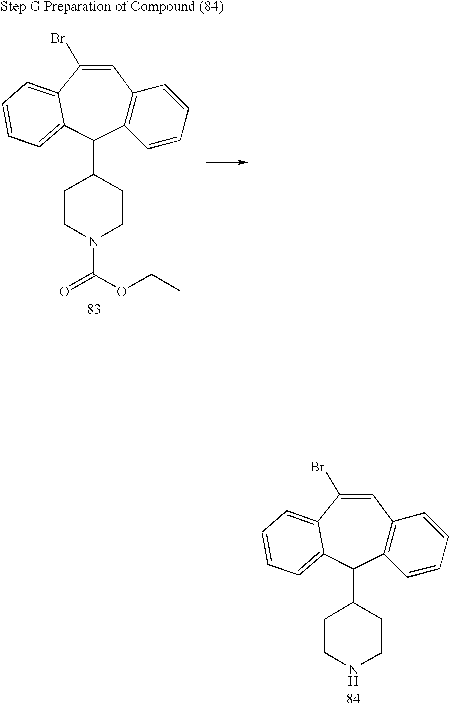

The product from Preparative Example 23, Step E was dissolved in 25 mL of conc. HCl and heated to reflux overnight. The reaction mixture was poured into ice basified with 50% w/w NaOH and then extracted with CH₂Cl₂ to give the desired compound (84).

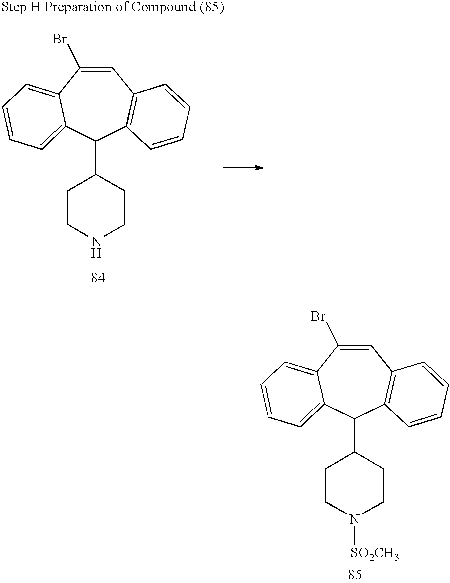

To a solution of the amine (84) (0.82 g) in CH₂Cl₂ (10 ml) was added triethyl amine (1.0 ml). Slowly, methane sulfonyl chloride (0.3 ml) was added and the mixture stirred over night at room temperature. To the reaction was added saturated sodium bicarbonate and then it was extracted with CH₂Cl₂. The organic layer was dried over magnesium sulfate, filtered and concentrated to dryness to give the desired compound (85).

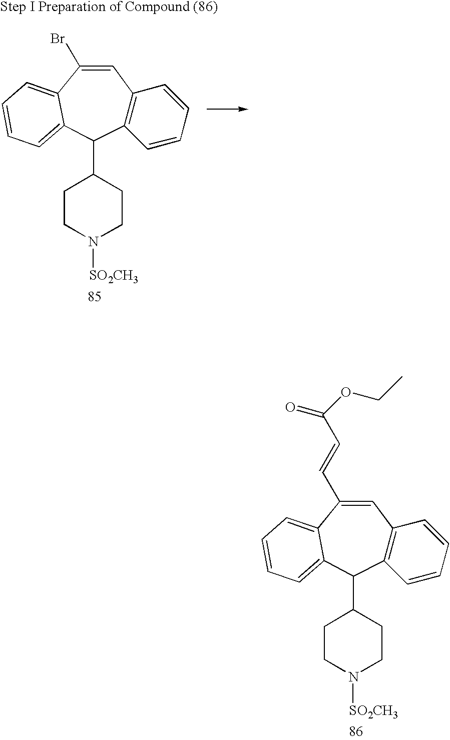

Compound (85) from Preparative Example 23 Step H, was dissolved in DMF. Successively, Et₃N (29 eq.), Ethyl acrylate (5.4 eq.), K₂CO₃ (5 eq.), Bu₄NBr (2 eq.) and Palladuim (II) acetate (0.13 eq.) were added. The mixture was stirred and heated to 100° C. for 4 h. After cooling, the mixture was concentrated and the residue was taken up in CH₂Cl₂ and extracted with CH₂Cl₂/H₂O. The organic layer was dried over Na₂SO₄ then concentrated and the residue purified by normal phase silica gel using 25% EtOAc-75% hexane to afford the title compound (86).

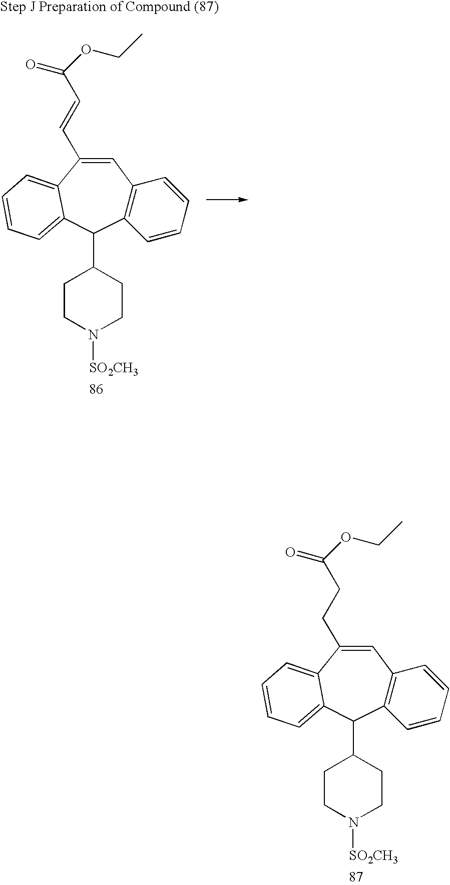

Compound (86) from Step I above was dissolved in EtOH, followed by the addition of CH₂Cl₂ at room temperature. To this reaction solution was added 10% w/w of PtO₂ and the reaction stirred at room temperature under H₂ (balloon) overnight. The reaction mixture was filtered through celite and concentrated to yield the title compound (87).

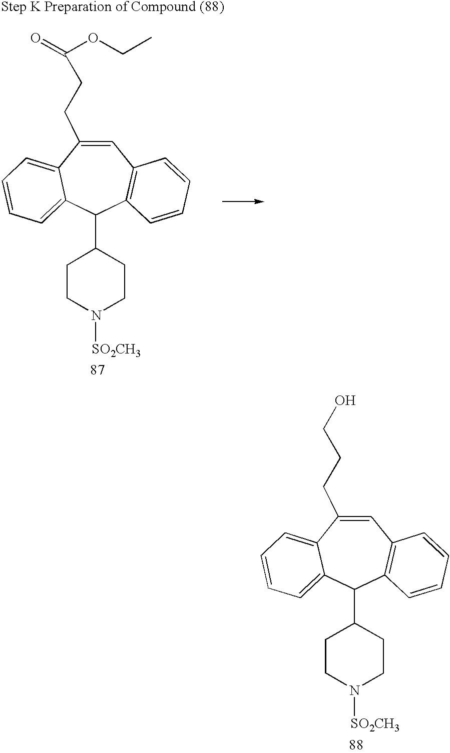

To a stirred solution of the ester (87) from Step J above, (0.63 g, 1.4 mmol) in THF (10 mL) at 0° C. was added a 1M solution of DIBAL (2.8 mL, 2.8 mmol). The resulting solution was stirred at room temperature overnight. An additional portion of 1M DIBAL was added and the mixture was stirred for 4 more hours at 0° C. The reaction solution was extracted with EtOAc-10% citric acid, 1N NaOH. The combined organic layer was dried over Na₂SO₄ filtered and evaporated to give title compound (88).

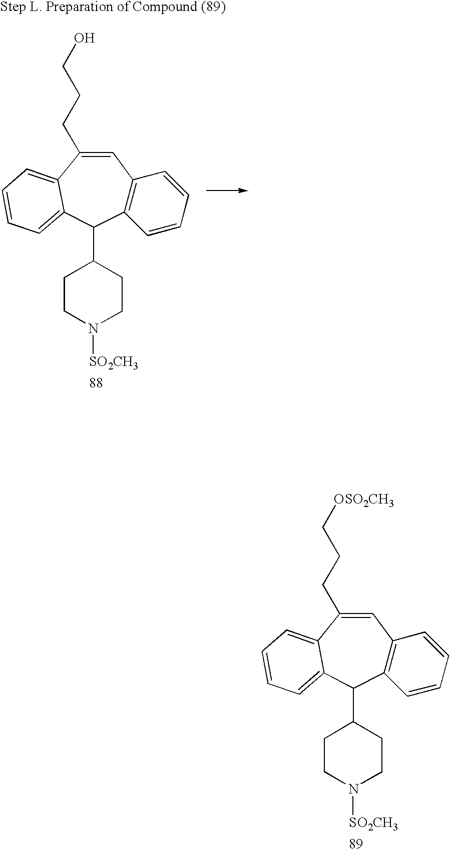

In a similar manner as was described in Preparative example 22, Step D, triethyl amine was added to a solution of Compound (88) in CH₂Cl₂ (100 ml). Slowly, methane sulfonyl chloride was added and the mixture stirred over night at room temperature. To the reaction was added saturated sodium bicarbonate and then it was extracted with CH₂Cl₂. The organic layer was dried over magnesium sulfate, filtered and concentrated to dryness. The crude product mixture was purified with column chromatography, eluting with 80% EtOAc—Hex, to afford the title compound (89).

### PREPARATIVE EXAMPLE 24

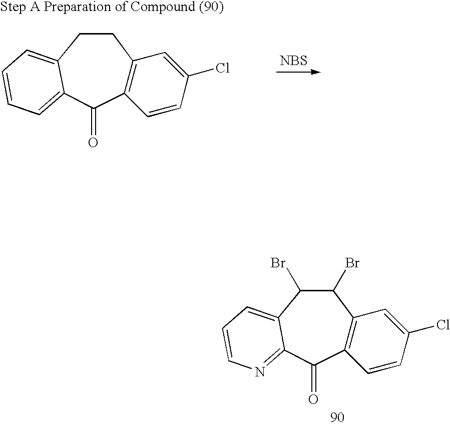

The tricyclic keto-compound (disclosed in U.S. Pat. No. 5,151,423) (30.0 g; 123.2 mmol) was combined with NBS (48.2 g; 271.0 mmol) and benzoyl peroxide (0.42 g) in CCl₄ (210 ml). The reaction was heated to 80° C. for 10 hr. The mixture was cooled and let stand for 8 hr. The resulting precipitate was filtered, added to MeOH (200 ml) and the resulting mixture was stirred over 2 days. The solid was filtered and dried under vacuum to a constant weight.

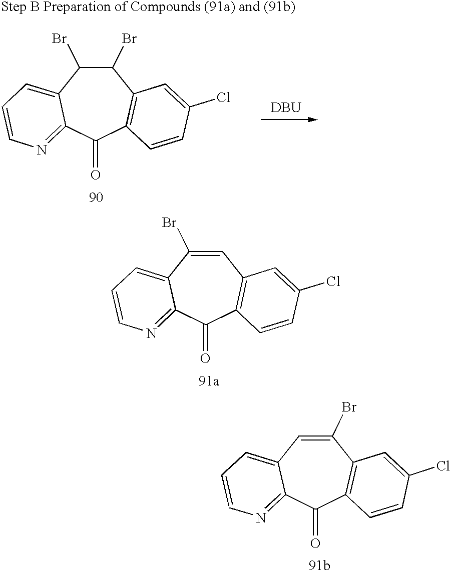

The dibromo compound (90) from Step A (35.72 g; 88.97 mmol) above was dissolved in CH₂Cl₂ (1.5 L) and cooled to 0° C. Dropwise, DBU (15.96 ml) was added and the suspension stirred for 3 hr. The reaction mixture was concentrated redissolved in CH₂Cl₂ (1.5 L) filtered through a bed of silica gel and rinsed with 5% EtOAc/CH₂Cl₂ (4 L). The combined rinses were concentrated and purified by flash silica gel column chromatography into pure 5 and 6 mono-bromo substituted compounds eluting with 10-30% EtOAc/Hex, then 3%EtOAc/CH₂Cl₂.

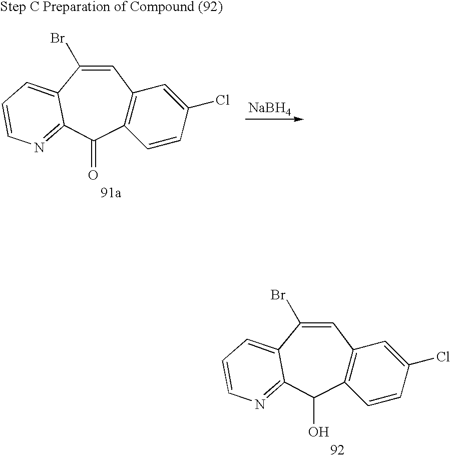

The 5-bromo substituted compound (91 a) from Step B above (4.0 g; 12.45 mmol) was taken up in MeOH and cooled to 0° C. NaBH₄ (916.4 mg; 24.2 mmol) was added and the reaction mixture stirred for 5.5 hr. The solvent was removed and the resulting residue was used directly.

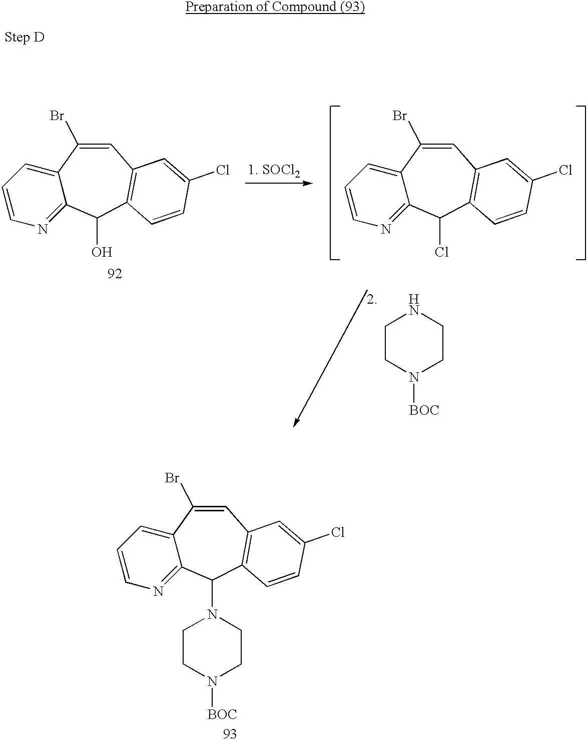

The alcohol compound (92) from Step C above (3.98 g; 12 mmol) was dissolved in CH₂Cl₂ cooled to 0° C. and treated with 2,6-Lutidine (5.73 ml; 49 mmol). SOCl₂ (1.8 ml; 24.6 mmol) was added and the reaction was allowed to stir and come to room temperature over 3 hr. The reaction mixture was poured into 0.5 N NaOH (80 ml) extracted and concentrated in vacuo. The crude product was taken up in CH₃CN and treated with 1,2,2,6,6-Pentamethylpiperidine (4.45 ml; 24.6 mmol) (Aldrich). The reaction was heated to 60-65° C. treated with tert-butyl 1-piperazinecarboxylate (2.32 g; 12 mmol) (Aldrich) and stirred over night under N₂ atmosphere. The reaction mixture was concentrated to dryness, redissolved in CH₂Cl₂ and washed with sat. aqueous NaCO₃. The organic layer was dried over Na₂SO₄, filtered and purified by flash silica gel column chromatography eluting with 1:4-1:2 EtOAc/Hexanes to afford the product as a solid.

### PREPARATIVE EXAMPLE 25

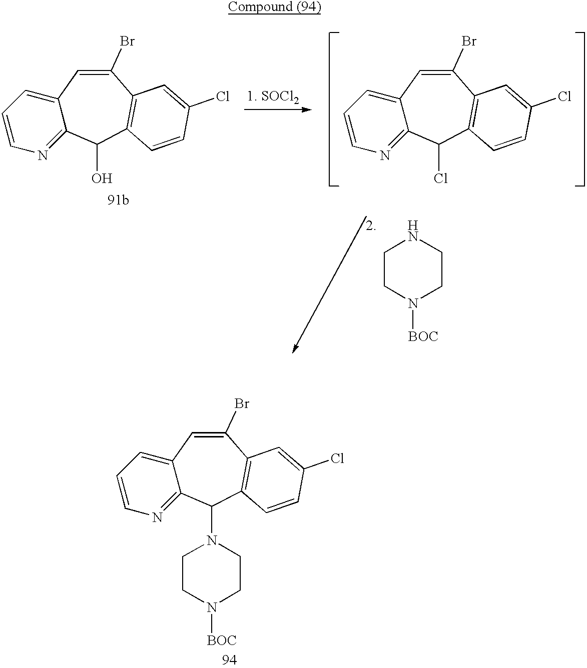

In essentially the same manner as in Preparative Example 24, Steps C-D, substituting the 6-Bromo substituted compound (91 b) from Step B, for the 5-Bromo substituted compound (91 a), Compound (94) was prepared (76.6 g, 100% yield).

### PREPARATIVE EXAMPLE 26

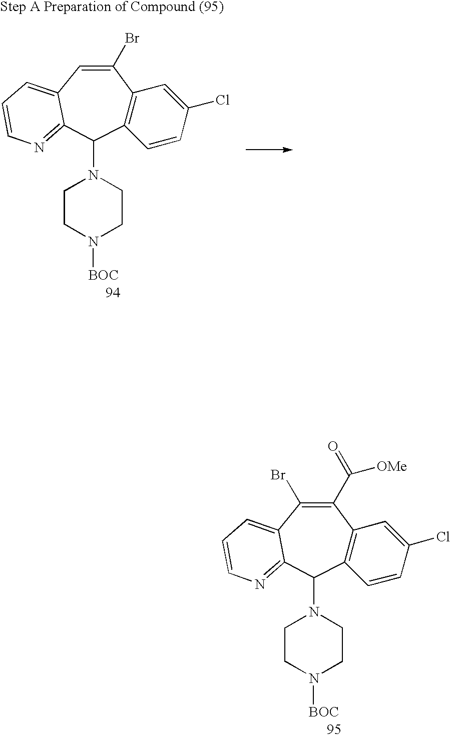

To a solution of Compound (94) from Preparative Example 25 (4.0 g, 8.16 mmol) in toluene (75 mL) and MeOH (20 mL), was added triphenyl phosphine (1.099 g, 4.08 mmol), DBU (1.7 g, 11.02 mmol) and palladium chloride (0.145 g, 0.82 mmol). The resulting solution was evacuated with CO at 100 psi and heated at 78° C.-82° C. for 5 hours, followed by the extraction with EtOAc-H₂O. The combined organic layer was then washed with brine, dried over Na₂SO₄, concentrated to dryness and purified by column chromatography, eluting with 30% EtOAc/70% Hexane to give Compound (95) (3.12 g, 100% yield, MH⁺=470.1).

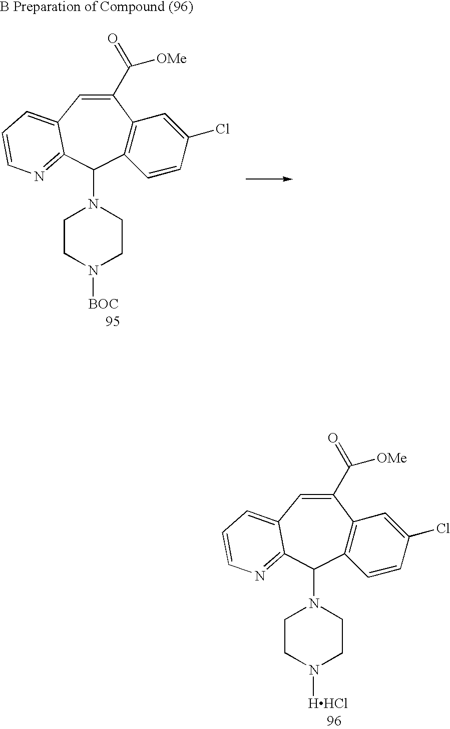

A solution of Compound (95) from Step A above (3.1 g, 6.6 mmol) in 4M HCl/Dioxane (120 mL) was stirred for 3 hours and then concentrated to dryness to give the crude salt of Compound (96) (3.89 g, 100% yield, MH⁺=370.2)

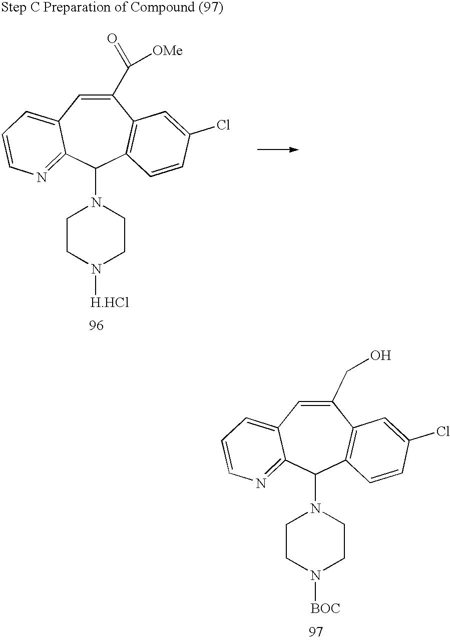

To a solution of Compound (96) from Step B above (3.43 g, 8.45 mmol) in THF (60 mL) at 0° C., was added DIBAL (7.21 g, 50.7 mmol). The resulting solution was warmed to room temperature, stirred overnight and then concentrated to dryness, followed by the addition of Boc anhydride (3.69 g, 16.9 mmol). The reaction was then extracted with CH₂Cl₂—H₂O, filtered over Na₂SO₄ and concentrated to dryness to afford Compound (97) (3.75 g, 100% yield, MH⁺=442.4).

Step C.1 Alternate Preparation of Compound (97)

A solution of compound 95 from Step A above (23.46 g, 50.98 mmol) in CH₂Cl₂—MeOH—H₂O (120 mL, 600 mL, 60 mL respectively) combined with LiOH (12.0 g, 350.88 mmol) was refluxed at 40° C. overnight. Solvent was removed from the reaction mixture. The residue was diluted with CH₂Cl₂ then acidified to pH 6 with 1N HCl. The organic layer was separated and washed with water, dried over Na₂SO₄ and concentrated. The product was dissolved in THF (285 mL) at 0° C. Triethyl amine (6 mL, 42.97 mmol) and ethyl chloroformate (4.1 mL, 42.97 mmol) were added and stirred at 0° C. for 1 h. The reaction mixture was filtered and the filtrate was cooled to −70° C. To this filtrate was added NaBH₄ (3.97 g, 104.94 mmol) and stirred for 1 h at −70° C. after which time 40 mL of MeOH was added dropwise. The solvents were removed and the residue taken up in methylene chloride, washed with sat. (aq) NaHCO₃, then brine, dried over Na₂SO₄ and concentrated to give Compound (97) as a solid.

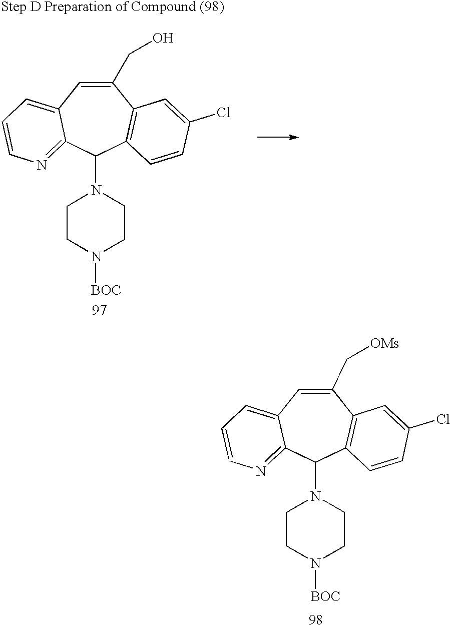

To a solution of Compound (97) from Step C above (3.74 g, 8.46 mmol) in CH₂Cl₂ (100 mL) was added triethyl amine (3.5 mL, 25.38 mmol) and methanesulfonyl chloride (1.45 g, 2.7 mmol). The resulting solution was stirred under nitrogen at room temperature for overnight and then washed with saturated NaHCO₃, then brine, and dried over Na₂SO₄ to give the mesylate compound (98) (3.86 g, 88% yield).

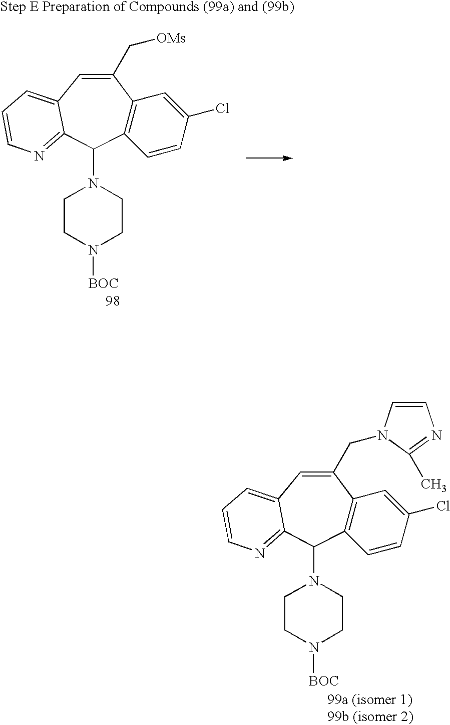

To a solution of 2-methylimidazole (2.43 g, 29.68 mmol) in DMF (30 mL) under N₂ was added NaH (0.53 g, 22.3 mmol) and stirred for 10 min, followed by the addition of Compound (98) from Step D above (3.86 g, 7.42 mmol). The solution was stirred over night. The solution was then concentrated to dryness and extracted with EtOAc-NaHCO₃, dried over Na₂SO₄, and concentrated. The crude product was purified by column chromatography, eluting with 2% MeOH—NH₃/98% CH₂Cl₂ to afford a mixture of isomers. Further separation was accomplished by Preparative HPLC Chiral AD Column chromatography, eluting with 25% IPA/75% hexane/0.2% DEA to give pure Compound (99a) (isomer 1) (0.160 g) and Compound (99b) (isomer 2) (0.140 g) (MH⁺=506.1).

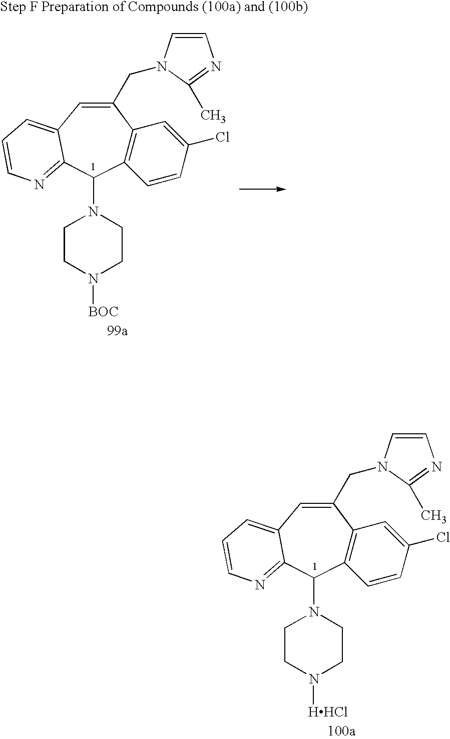

A solution of Compound (99a) (isomer 1) from Step E above (0.105 g, 0.21 mmol) in 4M HCl/Dioxane (10 mL) was stirred at room temperature for 3 hours and concentrated to dryness to afford Compound (100a) (0.147 g, 100% yield)

Compound (99b) (isomer 2) from Step E was treated in the same manner as isomer 1 above, to afford Compound (100b) (isomer 2).

### EXAMPLES 501 AND 502

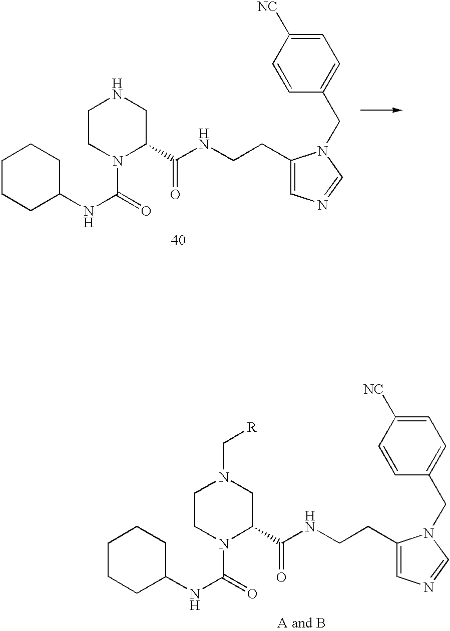

Sodium triacetoxyborohydride (75 mg, 0.336 mmol, 3.1 eq.) was added portion wise (3×25 mg) to a stirred solution of compound (40) (50 mg, 0.108 mmol, 1.0 eq.) from Preparative Example 11, Step B, and the appropriate aldehyde of the R group shown in the table below (0.336 mmol, 3.1 eq.), in a mixture of glacial acetic acid (0.5 ml) and anhydrous dichloromethane (10 ml) at 0° C. under a nitrogen atmosphere. The mixture was slowly (3 h) warmed to room temperature and stirred for another 12 h. The volatiles were removed under vacuum at 30° C. The residue was taken up in 1N aqueous NaOH solution (10 ml) and extracted with dichloromethane (5×5 ml). The combined organic extracts were washed with brine (5 ml), dried over Na₂SO₄, filtered, and concentrated under vacuum at room temperature. The product was purified by preparative scale thin layer chromatography (using either CH₂Cl₂:2 N NH₃/MeOH=90:10 v/v or CH₃CN:2 N NH₃/MeOH=90:10 v/v as eluent) over silica gel to afford the compounds listed in the Table A below:

Reference: Abdel-Magid, A. F., Maryanoff, C. A., Carson, K. G. Tetrahedron Lett. 1990, 31, 5595.

TABLE A

| Example # | R | Compound | Physical Data |
|---|---|---|---|
| 501 | 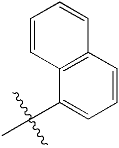 | A | MS: MH+ = 611.3 mp 100° C. (dec) 48% yield |
| 502 | 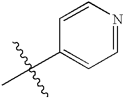 | B | MS: MH+ = 555.3 mp 80° C. (dec) 38% yield |

### EXAMPLE 503

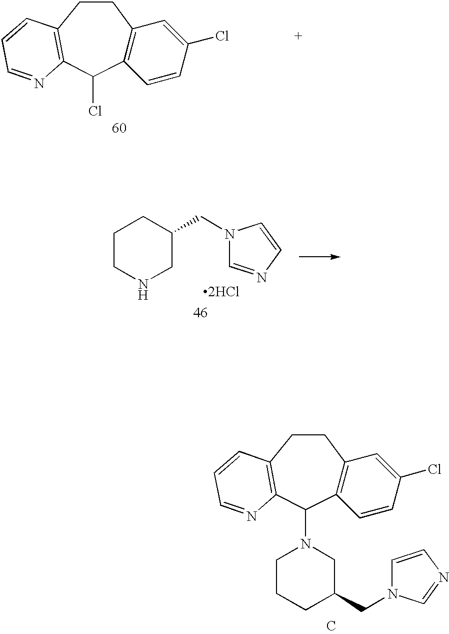

TEA (0.66 mL, 5.0 eq.) was added drop wise to a stirred solution of Compound (60) (0.25 g, 0.946 mmol) (see U.S. Pat. No. 5,151,423) and Compound (46) from Preparative Example 12, Step F (0.25 g, 1.1 eq.) in CH₂Cl₂ (5.0 mL). The resulting solution was stirred at room temperature for 24 h. then diluted with H₂O, neutralized with saturated NaHCO₃, and extracted with CH₂Cl₂. The combined organics were dried over Na₂SO₄, filtered, and concentrated under reduced pressure. The crude product was purified by flash chromatography using a 5% (10% NH₄OH in MeOH) in CH₂Cl₂ solution as eluent to yield Compound (C) as the first eluting isomer and a mixture of diastereomers:

Compound (C):LCMS: MH⁺=393; mp=71-75° C.; [α]_{D}²⁰=−65° (3.97 mg in 5.0 mL MeOH).

### EXAMPLE 504

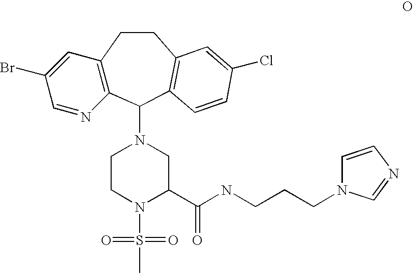

Compound (49) from Preparative Example 15, Step A, was reacted with Compound (52) from Preparative Example 17 using essentially the same procedure as in Example 503, to obtain Compound (O); FABMS (M+1)=623.

### EXAMPLE 505

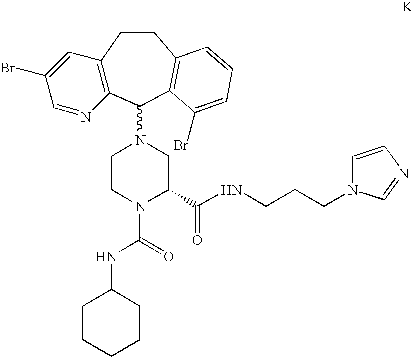

Compound (34) (0.9 gm) from Preparative Example 5 was reacted with compound (59) from Preparative Example 20, Step D 0.36 gm, 1 mmol) using essentially the same procedure as in Example 503, to obtain Compound (K); FABMS (M+1)=715.

### EXAMPLE 506

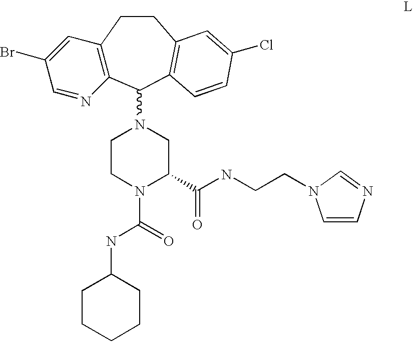

Compound (53) from Preparative Example 18 was reacted with Compound (49) from Preparative Example 15, Step A, using essentially the same procedure as in Example 503, to obtain Compound (L); FABMS (M+1)=656.

### EXAMPLE 507

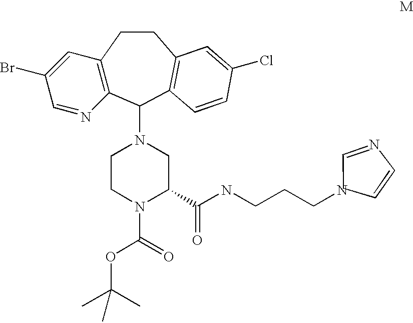

Compound (54)(0.9 gm) from Preparative Example 19 was reacted with Compound (49) from Preparative Example 15, Step A, using essentially the same procedure as in Example 503, to obtain Compound (M).

### EXAMPLE 508

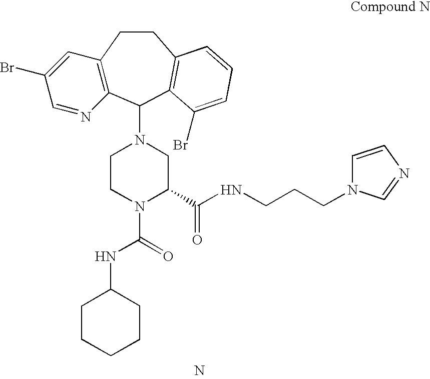

Compound (K) from Example 505 was further purified by HPLC on a Chiralcel AD column eluting with 30% Isopropanol/Hexanes: 0.2% DEA, to afford the pure isomer A Compound (N).

### EXAMPLE 509

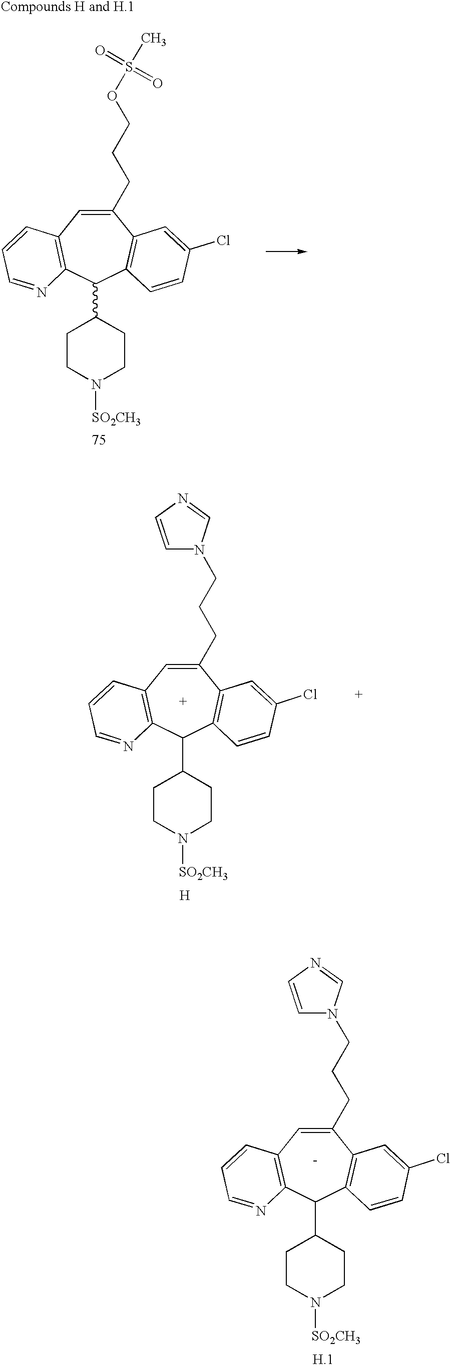

To a solution of compound (75) from Preparative example 22, Step D (0.2 g, 0.41 mmole) in DMF (7 ml) was added imidazolylsodium (0.054 g, 0.61 mmol). The reaction mixture was heated to 90° C. for 2 h then cooled and the DMF was removed. To the residue was added saturated sodium bicarbonate and the solution was extracted with CH₂Cl₂. The organic layer was dried over magnesium sulfate, filtered and concentrated to dryness. The crude product was purified by Biotage column chromatography eluting with 5% CH₃OH: (saturated with ammonia)-CH₂Cl₂, to afford the title compound as an enantiomeric mixture. The mixture was separated into pure enantiomers on Prep HPLC Chiral AD column eluting with 35-40% Isopropanol-Hexane: 0.2% Diethyl amine, to give the title compounds (H) and (H.1) MS 497 (MH⁺).

### EXAMPLE 510

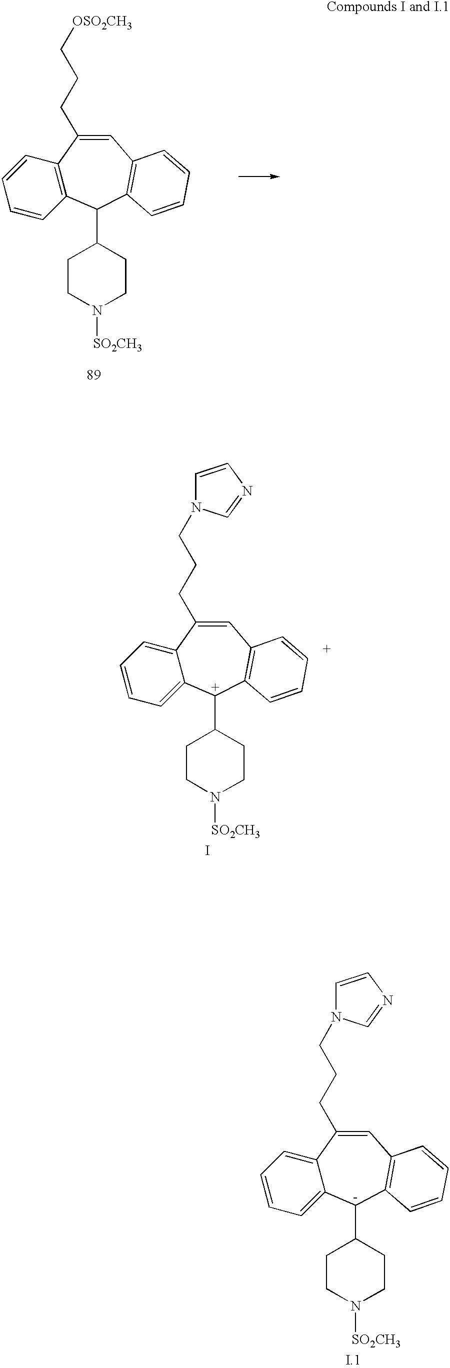

In a similar manner as described in Example 509 above, imidazolylsodium was added to a solution of the compound (89) from Preparative Example 23, Step L, in DMF (7 ml). The reaction mixture was heated to 90° C. for 2 h then cooled and the DMF was removed. To the residue was added saturated sodium bicarbonate and the solution was extracted with CH₂Cl₂. The organic layer was dried over magnesium sulfate, filtered and concentrated to dryness. The crude product was purified by Biotage column chromatography eluting with 5% CH₃OH: (saturated with ammonia)-CH₂Cl₂, to afford the title compound as an enantiomeric mixture. The mixture was separated into pure enantiomers on Prep HPLC Chiral AD column eluting with 35-40% Isopropanol-Hexane: 0.2% Diethyl amine, to give the title compounds (I) and (I.1) MS 497 (MH⁺).

### EXAMPLE 511

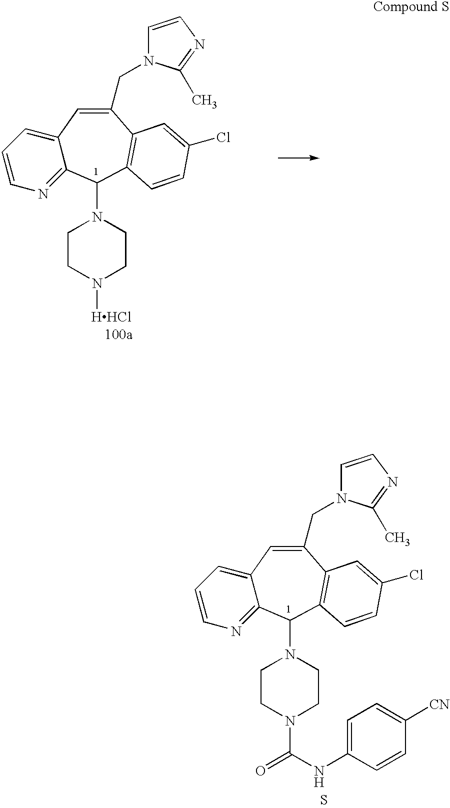

To a solution of compound 100a (1.3 g, 2.94 mmol) in CH₂Cl₂ (60 mL) was added triethyl amine (1.3 mL, 9.4 mmol) and p-cyano phenyl isocyanate (0.466 g, 3.24 mmol). The resulting solution was stirred at room temperature overnight, followed by the extraction with CH₂Cl₂ and saturated NaHCO₃ The organic layer was dried over Na₂SO₄, evaporated and the residue purified by column chromatography, eluting with 1%-2% MeOH—NH₃/98% CH₂Cl₂ to afford compound (S) (0.870 g, 48% yield) isomer 1; MS MH+=550.

### EXAMPLE 512

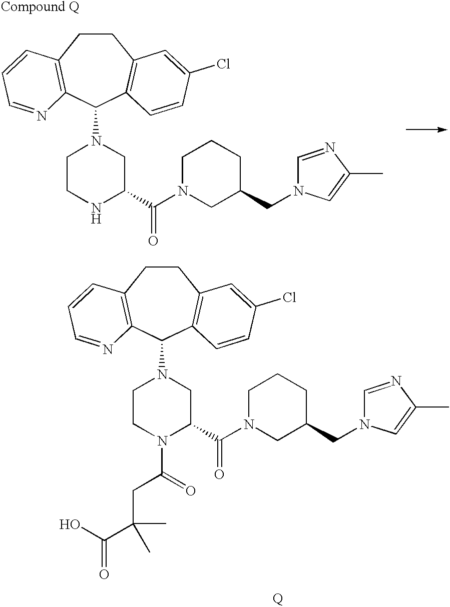

A mixture of the title compound (prepared as described in WO 00/37458; 0.206 g, 0.397 mmol), 2,2-dimethylsuccinic anhydride (0.081 g, 0.632 mmol, 1.6 eq.) and 1,2-dichloroethane (5.0 mL) was stirred at 25° C. for 15 hours. The mixture was concentrated in vacuo, diluted with CH₂Cl₂, washed with H₂O and the combined organic phase was dried over anhydrous Na₂SO₄, filtered, and concentrated. Purification preparative plate chromatography (silica) developing with 10% MeOH—CH₂Cl₂ afforded the product as a solid: 0.257 g, 100%; MS (CI) m/z 647 (MH⁺, 100%); mp=86.2° C.; [α]²⁰_{D}=−25.4° (2.30 mg/2 mL, CH₂Cl₂).

Compounds I, II, III, IV and V may be produced by processes known to those skilled in the art shown in the following reaction schemes.

Trypanosoma brucei FPTase SPA Assay

A competitive inhibition Scintillation Proximity Assay (SPA) was developed using Trypanosoma brucei FPT enzyme partially purified from Dr. Michael Gelb's lab (University of Washington, Seattle, Wash.). The TbFPT concentration was 4.7 mg/ml (BioRad) and estimated to be 1-5% pure based on SDS PAGE. The sample was provided in 30 mM potassium phosphate, pH 7.7, 30 mM NaCl, 1 mM DTT, 0.1% β-octylglucoside, and stored at −80° C.

Summary of the Final Reaction Conditions: The SPA Assay Buffer contained 50 mM Tris, 5 μM ZnCl₂, 5 mM MgCl₂, 0.01% Triton X-100, 5 mM DTT, pH 7.5. The reaction was performed using partially purified TbFPT (total protein)=0.005 μg/μl (equivalent to ˜0.4-2 nM FPT, 1-5% pure), for 60 mins at 30° C. The two substrates, biotin-CVIM and [³H]-FPP, were adjusted to concentrations of 0.25 μM each. FPP was added to FPT followed by the addition of each inhibitor. Inhibitors were two-fold serial diluted and added to the FPT reactions from DMSO stocks to give a final concentration of 2% DMSO and a starting inhibitor concentration of 20 μM. The reaction was initiated by adding the peptide.

Trypanosoma brucei Cell-Based Assay The Trypanosoma brucei mammalian bloodstream form was grown to a cell density of ˜5×10⁴ cells/ml for 120 hrs in a 24-well tissue culture plate in 1 ml of HMI-9 media. The media contained penicillin (100 U/ml), streptomycin (100 μg/ml) and 10% fetal bovine serum. The cells were grown at 37° C. with 5% CO₂. Inhibition studies were performed by adding compounds, in the presence of DMSO, to the culture. The final DMSO content was less than 0.5% and controls were performed to show that this level of DMSO did not inhibit cell growth. Cell growth and inhibition were assayed by counting the number of viable cells using a hemacytometer. The ED50 was determined as the concentration of inhibitor required to reduce the cell number relative to the control culture by 2-fold.

Compounds of the invention have a TbFPTase IC50 range of between 0.0019 μM to 15 μM, and a Tb BSF ED50 range of between 0.2 μM to <10 μM.

### Administration and Dosage

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. Examples of liquid forms are water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations, which are intended to be converted, shortly before use, to liquid form preparations for either oral or parenteral administration. Such liquid forms include solutions, suspensions and emulsions.

The compounds of the invention may be deliverable transdermally. The transdermal compositions can take the form of creams, lotions, aerosols and/or emulsions and can be included in a transdermal patch of the matrix or reservoir type as are conventional in the art for this purpose.

Preferably, the compounds of the invention are administered orally. Pharmaceutical compositions can contain any one of the compounds A-U or of formulas I-V used in the methods of the invention in combination with one or more additional compounds, which are useful in preventing or treating Trypanosoma brucei infection and/or reverse Trypanosoma brucei resistance. Additional compounds include but are not limited to pentamidine isethionate, suramine sodium, melarsoprol and eflornithine and/or compounds which inhibit multidrug resistance, e.g. tetrandrine.

Preferably, the pharmaceutical preparation is in a unit dosage form. In such form, the preparation is subdivided into unit doses containing therapeutically effective amounts of the active component, i.e., amounts that inhibit Trypanosoma brucei.

The compounds of this invention are administered to a patient in need of such treatment (e.g. a mammal, such as a human being) in an effective amount, e.g. a therapeutically effective amount, or a Trypanosoma brucei inhibitory effective amount. An effective amount is that amount necessary to inhibit FPT, and thereby inhibit the Trypanosoma brucei parasite.

The quantity of active compound in a unit dose of preparation may be varied or adjusted from about 1.0 mg to about 4500 mg, preferably from about 10.0 mg to about 1000 mg, according to the particular application.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day i.e., the total daily dosage may be divided and administered in portions up to two to four times over a 24 hour period.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 10 mg/day to about 9000 mg/day, in two to four divided doses.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A method for treating Trypanosoma brucei infections comprising administering to a patient in need of such treatment an effective amount of a Farnesyl Protein Transferase inhibitor selected from: 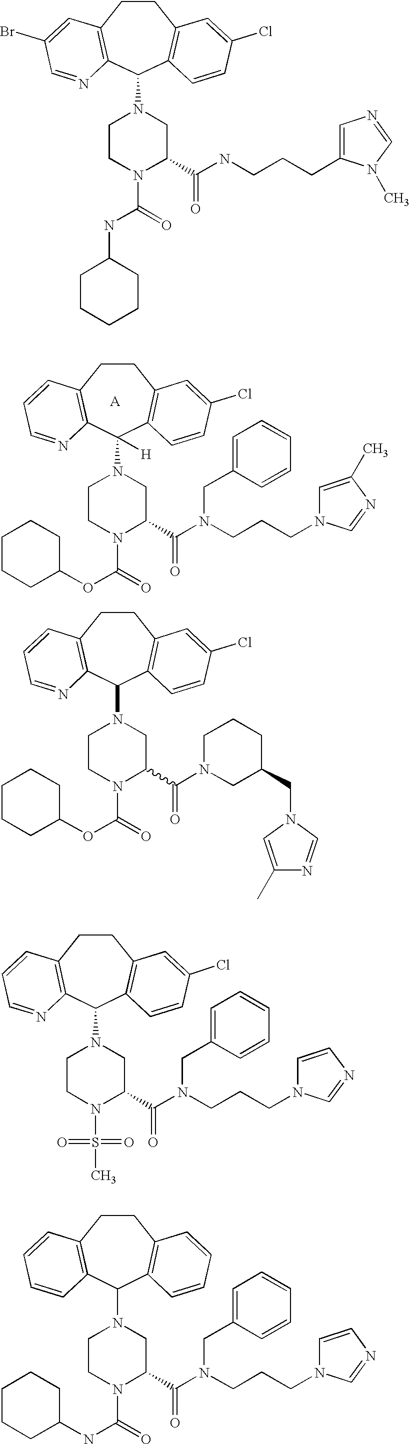 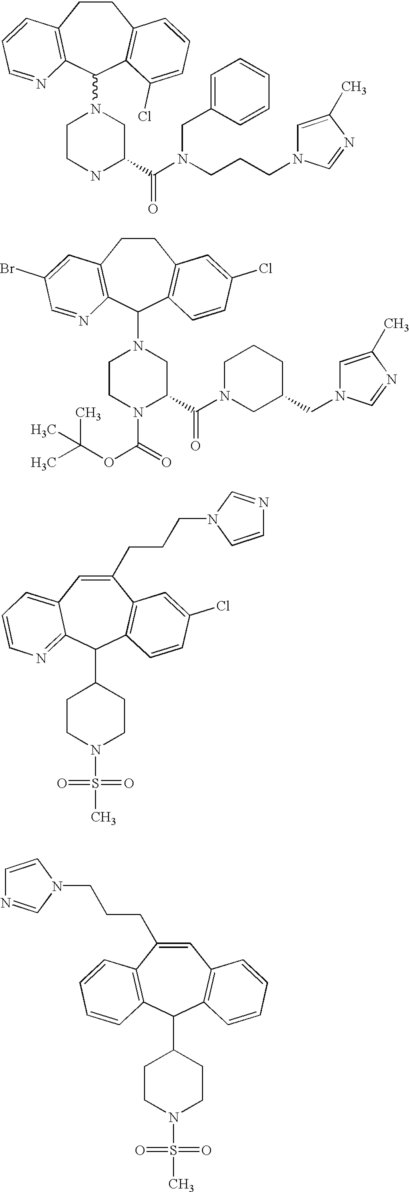 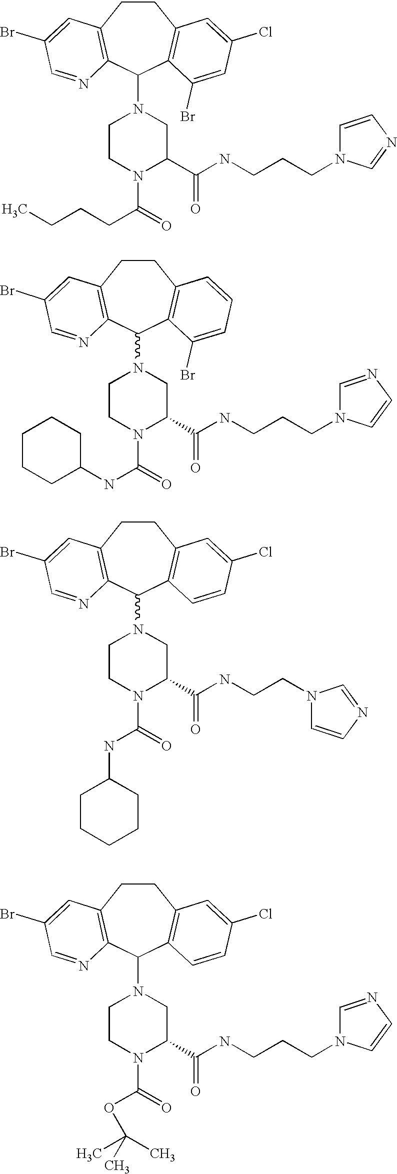 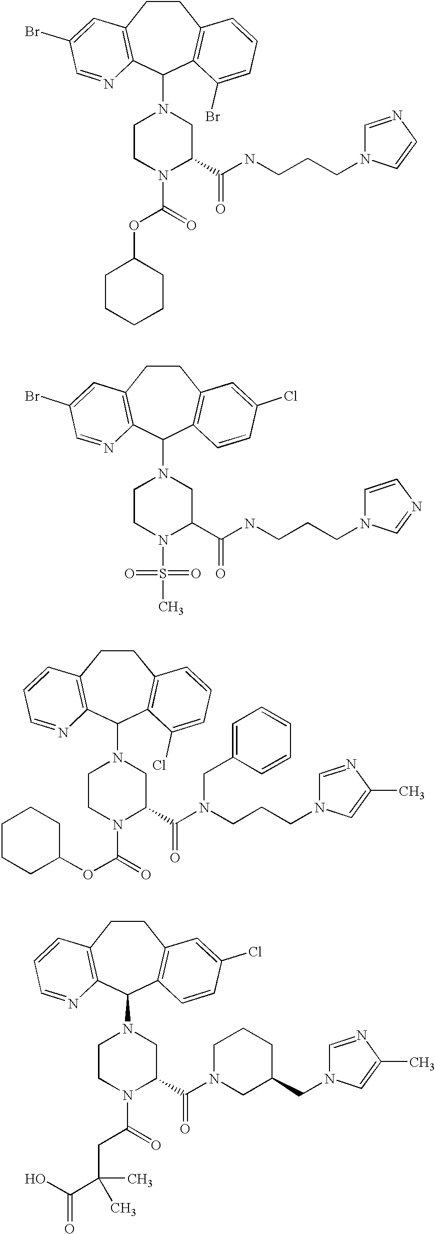 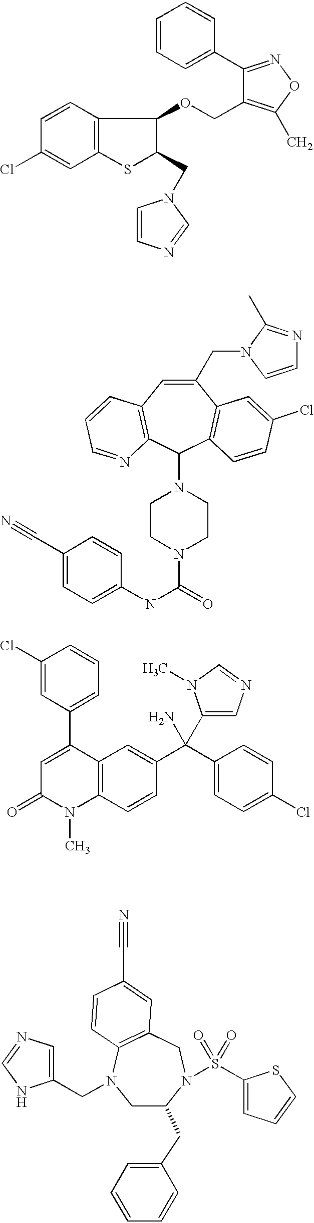
or a pharmaceutically acceptable salt or solvate thereof.

2. A method for treating Trypanosoma brucei infections comprising administering to a patient in need of such treatment an effective amount of a compound of the formulas I, II, III or IV 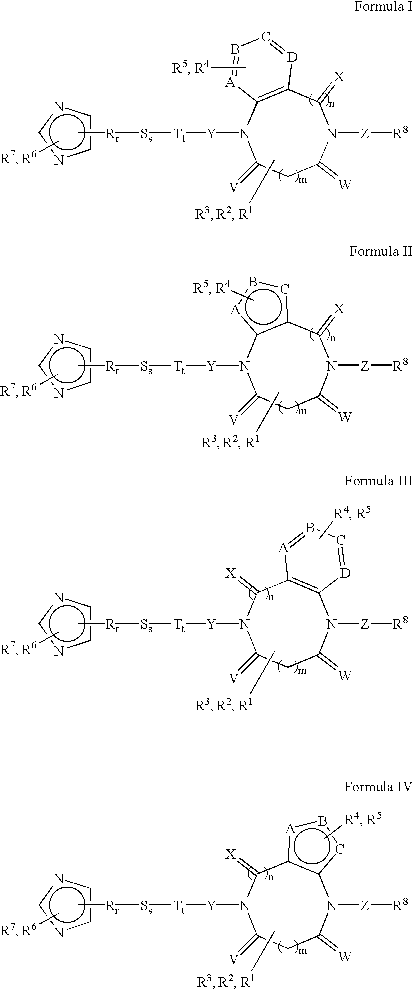
or their pharmaceutically acceptable salts or solvates thereof, wherein:
m, n, r, s and t are 0 or 1;
p is 0, 1 or 2;
V, W and X are selected from the group consisting of oxygen, hydrogen, R¹, R² or R³;
Z and Y are selected from the group consisting of CHR⁹, SO₂, SO₃, CO, CO₂, O, NR¹⁰, SO₂NR¹¹, CONR¹², 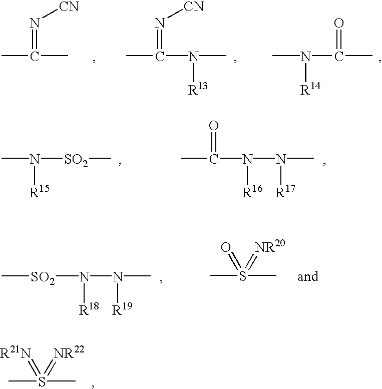
or Z may be absent;
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²⁴, R²⁵, R²⁶, R²⁷, R²⁸, R²⁹, R³⁰, R³¹, R³², R³³, R³⁴, R³⁵, R³⁶, R³⁷, and R³⁸ are selected from the group consisting of hydrogen, lower alkyl, substituted alkyl, aryl, or substituted aryl;
R⁴, R⁵ are selected from the group consisting of hydrogen, halo, nitro, cyano and U-R²³;
U is selected from the group consisting of sulfur, oxygen, NR²⁴, CO, SO, SO₂, CO₂, NR²⁵CO₂, NR²⁶CONR²⁷, NR²⁸SO₂, NR²⁹SO₂NR³⁰, SO₂NR³¹, NR³²CO, CONR³³, PO₂R³⁴ and PO₃R³⁵ or U is absent;
R¹, R², and R³ are selected from the group consisting of hydrogen, alkyl, alkoxycarbonyl, substituted alkyl, alkenyl, substituted alkenyl, alkynyl, substituted alkynyl, aralkyl, cycloalkyl, aryl, substituted aryl, heterocyclo, substituted heterocyclo, cyano, carboxy, carbamyl (e.g. CONH₂) or substituted carbamyl further selected from CONH alkyl, CONH aryl, CONH aralkyl or cases where there are two substituents on the nitrogen selected from alkyl, aryl or aralkyl; R⁸ and R²³ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo, substituted heterocyclo;
any two of R¹, R² and R³ can be joined to form a cycloalkyl group;
R, S and T are selected from the group consisting of CH₂, CO and CH(CH₂)pQ wherein Q is NR³⁶R³⁷, OR³⁸, or CN; and
A, B, C and D are carbon, oxygen, sulfur or nitrogen with the provisos that
1) when m is zero then V and W are not both oxygen or
2) W and X together can be oxygen only if Z is either absent, O, NR¹⁰, CHR⁹, 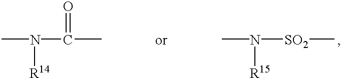
in formulas I and II, and V and X together can be oxygen only if Y is O, NR¹⁰, CHR⁹, 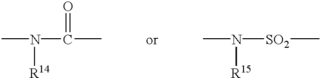
in formulas III and IV or
3) R²³ may be hydrogen except when U is SO, SO₂, NR²⁵CO₂ or NR²⁸SO₂, or
4) R⁸ may be hydrogen except when Z is SO₂, CO₂, or 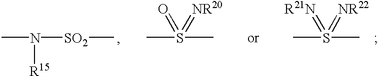
R³⁹ is halo, trifluoromethyl, trifluoromethoxy, hydroxy, alkoxy, cycloalkoxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl; alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido, substituted sulfonamido, nitro, cyano, carboxy, CON(R⁴⁴)₂, where the two R⁴⁴ moieties can be the same or different, alkoxycarbonyl, aryl, substituted aryl, guanidine or heterocyclo;
R⁴⁰ is halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino or heterocyclo;
R⁴¹ is halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino or heterocyclo;
R⁴² is alkyl, substituted alkyl, halo, trifluoromethoxy, trifluoromethyl, hydroxy, alkoxy, cycloalkoxy, heterocyclooxy, alkanoyl, alkanoyloxy, amino, alkylamino, aralkylamino, cycloalkylamino, heterocycloamino, dialkylamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido or aryloxy;
R⁴³ is alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different each being independently selected from the group consisting of hydrogen, alkyl, aryl, and aralkyl; and
R⁴⁴ is alkyl, aryl or aralkyl.

3. A method for treating Trypanosoma brucei infections comprising administering to a patient in need of such treatment an effective amount of a compound of the formula V: 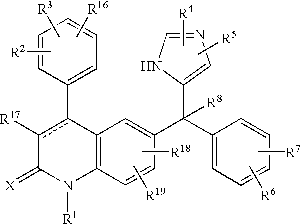
or a pharmaceutically acceptable salt or solvate thereof
wherein:
the dotted line represents an optional bond;
X is oxygen or sulfur;
R¹ is hydrogen, alkyl, Ar¹, Ar²alkyl, quinolinylalkyl, pyridylalkyl, hydroxyalkyl, alkoxyalkyl, aminoalkyl; or a radical of formula -Alk¹-C(═O)R⁹, -Alk¹-S(O)R⁹ or -Alk¹-S(O)₂—R⁹, wherein Alk¹ is alkanediyl, R⁹ is hydroxy, alkyl, alkoxy, amino, alkylamino or alkylamino substituted with alkoxycarbonyl;
R², R³ and R¹⁶ each independently are hydrogen, hydroxy, halo, cyano, alkyl, alkoxy, hydroxyalkoxy, alkoxyalkoxy, aminoalkoxy, Ar¹, Ar²alkyl, Ar²oxy, Ar²alkoxy, hydroxycarbonyl, alkoxycarbonyl, trihalomethyl, trihalomethoxy, alkenyl, 4,4-dimethyloxazolyl; or when on adjacent positions R² and R³ taken together may form a bivalent radical of formula
—O—CH₂—O—
—O—CH₂—CH2-O—
—O—CH═CH—
—O—CH₂—CH₂
—O—CH₂—CH₂—CH₂
or
—CH═CH—CH═CH—
R⁴ and R⁵ each independently are hydrogen, halo, Ar¹, alkyl, hydroxyalkyl, alkoxyalkyl, alkoxy, alkylthio, amino, hydroxycarbonyl, alkoxycarbonyl, alkylS(O)alkyl or alkylS(O)₂ alkyl with the proviso that when R⁴ or R⁵ is bound to one of the nitrogen atoms in the imidazole ring, the hydrogen on the nitrogen is replaced by R⁴ or R⁵ wherein R⁴ and R⁵ is selected from the group consisting of hydrogen, Ar¹, alkyl, hydroxyalkyl, alkoxyalkyl, alkoxycarbonyl, alkylS(O)alkyl and alkylS(O)₂alkyl;
R⁶ and R⁷ each independently are hydrogen, halo, cyano, alkyl, alkoxy, Ar²oxy, trihalomethyl, alkylthio, alkylamino, or when on adjacent positions R⁶ and R⁷ taken together may form a bivalent radical of formula —O—CH₂—O— or —CH═CH—CH═CH—;
R⁸ is hydrogen, alkyl, cyano, hydroxycarbonyl, alkoxycarbonyl, alkylcarbonylalkyl, cyanoalkyl, alkoxycarbonylalkyl, carboxyalkyl, hydroxyalkyl, aminoalkyl, imidazolyl, haloalkyl, alkoxyalkyl, aminocarbonylalkyl, or a radical of formula —O—R¹⁰, —S—R¹⁰, —N—R¹¹R¹² wherein
R¹⁰ is hydrogen, alkyl, alkylcarbonyl, Ar¹, Ar²alkyl, alkoxycarbonylalkyl, or a radical or formula -Alk²-OR¹³ or -Alk²-NR¹⁴R¹⁵;
R¹¹ is hydrogen, alkyl, Ar¹ or Ar²alkyl;
R¹² is hydrogen, alkyl, alkylcarbonyl, alkoxycarbonyl, alkylaminocarbonyl, Ar¹, Ar²alkyl, alkylcarbonylalkyl, an amino acid, Ar¹carbonyl, Ar²alkylcarbonyl, aminocarbonylcarbonyl, alkoxyalkylcarbonyl, hydroxy, alkoxy, aminocarbonyl, di(alkyl)aminoalkylcarbonyl, amino, alkylamino, alkylcarbonylamino, or a radical or formula -Alk²-OR¹³ or -Alk²-NR¹⁴R¹⁵;
wherein
Alk² is alkanediyl;
R¹³ is hydrogen, alkyl, alkylcarbonyl, hydroxyalkyl, Ar¹ or Ar²alkyl;
R¹⁴ is hydrogen, alkyl, Ar¹ or Ar²alkyl;
R¹⁵ is hydrogen, alkyl, alkylcarbonyl, Ar¹ or Ar²alkyl;
R¹⁷ is hydrogen, halo, cyano, alkyl, alkoxycarbonyl, Ar¹;
R¹⁸ is hydrogen, alkyl, alkoxy or halo;
R¹⁹ is hydrogen or alkyl;
Ar¹ is phenyl or phenyl substituted with alkyl, hydroxy, amino, alkoxy or halo; and
Ar² is phenyl or phenyl substituted with alkyl, hydroxy, amino, alkoxy or halo.

4. A method for treating Trypanosoma brucei infection comprising administering to a patient in need of such treatment an effective amount of a compound used in the method of claim 1, in combination with an effective amount of an additional anti-Trypanosoma brucei agent and/or an additional agent for reversing anti-Trypanosoma brucei resistance.

5. A method for treating Trypanosoma brucei infection comprising administering to a patient in need of such treatment an effective amount of the compound used in the method of claim 2, in combination with an effective amount of an additional anti-Trypanosoma brucei agent and/or an additional agent for reversing anti-Trypanosoma brucei resistance.

6. A method for treating Trypanosoma brucei infection comprising administering to a patient in need of such treatment an effective amount of the compound used in the method of claim 3, in combination with an effective amount of an additional anti-Trypanosoma brucei agent and/or an additional agent for reversing anti-Trypanosoma brucei resistance.

7. The method of claim 4, wherein said compound is administered prior to, concurrent to or subsequent to the administration of said additional anti-Trypanosoma brucei agent and/or an additional agent for reversing anti-Trypanosoma brucei resistance.

8. The method of claim 4 wherein said additional anti-Trypanosoma brucei agent is selected from the group consisting of:
a) pentamidine isethionate;
b) suramine sodium;
c) melarsoprol and
d) eflornithine.

9. The method of claim 4 wherein said agent for reversing anti-Trypanosoma brucei resistance is an inhibitor of multidrug resistance.

10. The method of claim 5, wherein said compound is administered prior to, concurrent to or subsequent to the administration of said additional anti-Trypanosoma brucei agent and/or an additional agent for reversing anti-Trypanosoma brucei resistance.

11. The method of claim 5 wherein said additional anti-Trypanosoma brucei agent is selected from the group consisting of:
a) pentamidine isethionate;
b) suramine sodium;
c) melarsoprol and
d) eflornithine.

12. The method of claim 5 wherein said agent for reversing anti-Trypanosoma brucei resistance is an inhibitor of multidrug resistance.

13. The method of claim 6, wherein said compound is administered prior to, concurrent to or subsequent to the administration of said additional anti-Trypanosoma brucei agent and/or an additional agent for reversing anti-Trypanosoma brucei resistance.

14. The method of claim 6 wherein said additional anti-Trypanosoma brucei agent is selected from the group consisting of:
a) pentamidine isethionate;
b) suramine sodium;
c) melarsoprol and
d) eflornithine.

15. The method of claim 6 wherein said agent for reversing anti-Trypanosoma brucei resistance is an inhibitor of multidrug resistance.

16. A pharmaceutical composition comprising an effective amount of a compound used in the method of claim 1, and a pharmaceutically acceptable carrier.

17. A pharmaceutical composition comprising an effective amount of the compound used in the method of claim 2, and a pharmaceutical acceptable carrier.

18. A pharmaceutical composition comprising an effective amount of the compound used in the method of claim 3, and a pharmaceutical acceptable carrier.

19. A pharmaceutical composition comprising an effective amount of
(1) a compound used in the method of claim 1;
(2) an additional anti-Trypanosoma brucei agent; and/or
(3) an agent for reversing anti-Trypanosoma brucei resistance.

20. The composition of claim 19 wherein said additional anti-Trypanosoma brucei agent is selected from the group consisting of:
a) pentamidine isethionate;
b) suramine sodium;
c) melarsoprol and
d) eflornithine.

21. The composition of claim 19 wherein said agent for reversing anti-Trypanosoma brucei resistance is an inhibitor of multidrug resistance.

22. A pharmaceutical composition comprising an effective amount of
(1) the compound used in the method of claim 2;
(2) an additional anti-Trypanosoma brucei agent; and/or
(3) an agent for reversing anti-Trypanosoma brucei resistance.

23. The composition of claim 22 wherein said additional anti-Trypanosoma brucei agent is selected from the group consisting of:
a) pentamidine isethionate;
b) suramine sodium;
c) melarsoprol and
d) eflornithine.

24. The composition of claim 22 wherein said agent for reversing anti-Trypanosoma brucei resistance is an inhibitor of multidrug resistance.

25. A pharmaceutical composition comprising an effective amount of
(1) the compound used in the method of claim 3;
(2) an additional anti-Trypanosoma brucei agent; and/or
(3) an agent for reversing anti-Trypanosoma brucei resistance.

26. The composition of claim 25 wherein said additional anti-Trypanosoma brucei agent is selected from the group consisting of:
a) pentamidine isethionate;
b) suramine sodium;
c) melarsoprol and
d) eflornithine.

27. The composition of claim 25 wherein said agent for reversing anti-Trypanosoma brucei resistance is an inhibitor of multidrug resistance.

28. The method of claim 2 wherein said compound is

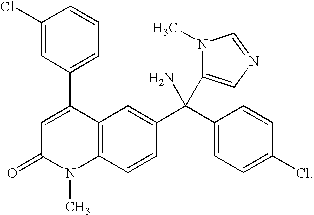

29. The method of claim 3 wherein said compound is [chemical structure]
